(19) United States
(12) Patent Application Publication
(10) Pub. No.: US 2024/0301082 A1
(43) Pub. Date: Sep. 12, 2024

(54) ANTI-CD73 COMPOUNDS TO TREAT ONCOVIRUS-POSITIVE CANCERS

(71) Applicant: Corvus Pharmaceuticals, Inc., Burlingame, CA (US)

(72) Inventors: Richard A. Miller, Burlingame, CA (US); Craig Hill, Burlingame, CA (US)

(21) Appl. No.: 18/289,261

(22) PCT Filed: May 2, 2022
(86) PCT No.: PCT/US2022/027291

§ 371 Date: Nov. 2, 2023

Related U.S. Application Data
(60) Provisional application No. 63/184,660, filed on May 5, 2021.

(51) Int. Cl. C07K 16/28; A61K 39/00

(52) U.S. Cl. CPC C07K 16/2896; A61K 2039/505; C07K 2317/24; C07K 2317/55; C07K 2317/565; C07K 2317/622; C07K 2317/92

(57) ABSTRACT

Provided herein are, inter alia, methods for treating oncovirus-positive cancers using anti-CD73 compounds, such as anti-CD73 antibodies. Exemplary oncovirus include human papilloma virus, hepatitis, cytomegalovirus, Epstein-Barr virus, human immunodeficiency virus, human herpes virus-8, polyomavirus, adenovirus, human T-cell leukemia virus, and the like.

### CROSS-REFERENCE TO RELATED APPLICATIONS

This application claims the benefit of priority to U.S. Application No. 63/184,660 filed May 5, 2021, the disclosure of which is incorporated by reference herein in its entirety.

### REFERENCE TO A “SEQUENCE LISTING,” A TABLE, OR A COMPUTER PROGRAM LISTING APPENDIX SUBMITTED AS AN ASCII FILE

The Sequence Listing written in file 048517-553001WO_SL_ST25.txt, created Apr. 29, 2022, 10,048 bytes, machine format IBM-PC, MS Windows operating system, is hereby incorporated by reference.

### BACKGROUND

The glycosyl-phosphatidylinositol-anchored CD73 antigen is considered the rate-limiting enzyme in the generation of extracellular adenosine (Stagg J, Smyth M J. Extracellular adenosine triphosphate and adenosine in cancer. Oncogene. 2010; 29:5346-58). CD73 can be found constitutively expressed at high levels on various types of cancer cells. CD73-generated adenosine is assumed to suppress adaptive anti-tumor immune responses thereby promoting tumor growth and metastasis. There is a need in the art for antibody-based CD73 cancer therapy which inhibits the catalytic activity of CD73, thereby blocking adenosinse production and relieving adenosine-mediated immunosuppression. This disclosure addresses these and other needs in the art.

### BRIEF SUMMARY

Provided herein are methods of treating oncovirus positive cancers in a patient by administering to the patient an effective amount of an anti-CD73 compound. In embodiments, the methods comprises detecting an oncovirus in a cancer cell obtained from the patient. In embodiments, the oncovirus is human papilloma virus (HPV), hepatitis (e.g., hepatitis B virus, hepatitis C virus), cytomegalovirus (CMV) Epstein-Barr virus (EBV), human immunodeficiency virus (HIV), human herpes virus-8 (HHV-8 or Kaposi's sarcoma-associated herpesvirus or KSHV), a polyomavirus (e.g., merkel cell, John Cunningham, BK), an adenovirus, human T-cell leukemia virus type-1 (HTLV-1), or human T-cell leukemia virus type-2 (HTLV-2). In embodiments, the cancer is head and neck cancer (e.g., oral cavity cancer, oropharyngeal cancer, laryngeal cancer, throat cancer), cervical cancer, penile cancer, vaginal cancer, anal cancer, vulvar cancer, bladder cancer, breast cancer, liver cancer, stomach cancer, colorectal cancer, pancreatic cancer, lymphoma (e.g., Burkitt lymphoma, Hodgkin lymphoma, non-Hodgkin lymphoma, primary effusion lymphoma, cutaneous T-cell lymphoma), colon cancer, glioma, prostate cancer, salivary gland cancer, neuroblastoma, testicular cancer, Kaposi's sarcoma, merkel cell carcinoma, gastrointestinal cancer, urogenital tract cancer, rhabdomyosarcoma, brain cancer (e.g., glioblastoma, oligodendroglioma, ependymomas), or adult T-cell leukemia/lymphoma. These and other embodiments are described in detail herein.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, N Y 1989). Any methods, devices and materials similar or equivalent to those described herein can be used in the practice of this disclosure. The following definitions are provided to facilitate understanding of certain terms used frequently herein and are not meant to limit the scope of the present disclosure.

An “anti-CD73 compound” refers to any compound (e.g., small molecule, peptide, protein, antibody) capable of binding to CD73 or otherwise inhibiting the ability of CD73 to perform normal functions in the adenosine pathway. In embodiments, the anti-CD73 compound is an anti-CD73 antibody. Exemplary anti-CD73 antibodies include 1E9 antibodies, IgG1 antibodies, humanized 1E9 antibodies, humanized IgG1 antibodies, and the like. The anti-CD73 compounds described herein are immunostimulatory.

“CPI-006” or “CPX-006” or “mupadolimab” is a humanized CD73 antibody, wherein the light chain is SEQ ID NO:8 and the heavy chain is SEQ ID NO: 7, described herein. CPI-006 is also described in WO 2017/100670 where the heavy chain is SEQ ID NO:53, and the light chain is SEQ ID NO:55, which correspond to SEQ ID NO: 9 and SEQ ID NO: 10, respectively.

“Oleclumab” or “MEDI9447” as provided herein, refers to the anti-CD73 antibody described by CAS Number 1803176-05-7; FDA UNII Code: 5CRY01URYQ; and Hay et al, Oncoimmunology, 5:(8) (2016)).

“AD2” as provided herein refers the anti-CD73 antibody described by Borrione P et al. (“CD38 stimulation lowers the activation threshold and enhances the alloreactivity of cord blood T cells by activating the phosphatidylinositol 3-kinase pathway and inducing CD73 expression.” J Immunol 162:6238-46 (1999), which is hereby incorporated in its entirety and for all purposes.

The term “oncovirus” refers to a virus that causes a cancer. Exemplary virus that cause cancer include human papilloma virus (HPV), hepatitis (e.g., hepatitis B or C), cytomegalovirus (CMV) Epstein-Barr virus (EBV), human immunodeficiency virus (HIV), human herpes virus-8 (HHV-8), a polyomavirus (e.g., merkel cell, John Cunningham, BK), an adenovirus, human T-cell leukemia virus type-1 (HTLV-1), or human T-cell leukemia virus type-2 (HTLV-2)

The term “oncovirus-positive cancer” refers to a cancer in which an oncovirus is present in cancer cells and/or tumor microenvironment.

The term “human papilloma virus” or “HPV” refers to a human papilloma virus that is an oncovirus. Exemplary human papilloma virus that are an oncovirus include HPV-16, HPV-18, HPV-31, HPV-33, HPV-34, HPV-35, HPV-39, HPV-45, HPV-51, HPV-52, HPV-56, HPV-58, HPV-59, HPV-65, HPV-66, HPV-68, and HPV-69.

“B Cells” or “B lymphocytes” refer to their standard use in the art. B cells are a type of white blood cell (leukocyte) capable of developing into antibody-producing cells, also referred to herein as “mature B cell” or “differentiated B cell.” The terms a “differentiated B cell” or “mature B cell” includes antibody-secreting B cells (e.g., plasmablasts, plasma cells) as well as memory B cells, which present antibodies on their cell surface. Differentiated B cells reside in secondary lymphoid organs and are characterized by the specific Immunoglobulin (Ig) they express. In embodiments, the differentiated B cell expresses IgM, IgG, IgA, IgE or a combination thereof. In embodiments, the differentiated B cell expresses IgM. In embodiments, the differentiated B cell expresses IgG. In embodiments, the differentiated B cell expresses IgA. In embodiments, the differentiated B cell expresses IgE. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

The term “plasma cell” or “plasma B cell” is used in accordance with its plain and ordinary meaning and refers to white blood cells that secrete antibodies in response to antigens. The plasma cells originate through differentiation of B cells.

The term “memory B cell” is used in accordance with its plain and ordinary meaning and refers to B lymphocytes that form part of the adaptive immune system. Their function is to memorize the characteristics of the antigen that activated their parent B cell during initial infection such that if the memory B cell later encounters the same antigen, it triggers an accelerated and robust secondary immune response.

The term “B-cell activation” or “activating B cells” or “activate B cells” as provided herein are terms well known in the art and are used according to their conventional meaning in the art. For example, B cell activation involves binding of B cell receptors (BCRs) to an antigen, or T cells providing an activation signal to the B cell (thymus-dependent activation). Upon activation, B cells upregulate the expression of specific B cell activation marker proteins (e.g., CD69, CD83), which are characteristic of activated B cells.

The term “redistributing” or “redistribution” as provided herein refers to an effect a compound (e.g., anti-CD73 antibody as provided herein) has on the location of B cells in an organism. In embodiments, the compound (e.g., anti-CD73 antibody as provided herein) binds to CD73 expressing B cells and modulates the location of a B cell in an organism relative to a standard control. In embodiments, where the compound (e.g., anti-CD73 antibody as provided herein) modulates the location of a B cell in an organism, there are less B cells in the periphery (e.g., peripheral blood) of an organism relative to the absence of the compound. In embodiments, the amount of B cells (number of B cells) present in peripheral blood of the subject is less relative to the absence of the anti-CD73 antibody. In embodiments, the amount (number) of B cells present in primary lymphoid organs (e.g., spleen or thymus) of the subject is greater relative to the absence of the anti-CD73 antibody.

The term “immunostimulatory” as provided herein refers to the ability of the anti-CD73 compounds described herein to activate the immune system or increase activity of any of its components. In embodiments, “immunostimulatory” refers to the ability of the anti-CD73 compounds to activate B cells. In embodiments, “immunostimulatory” refers to the ability of the anti-CD73 compounds to increase inflammatory cytokines in the subject, wherein the inflammatory cytokines are TNF-α, TNF-β, MIP-1α, MIP-10, IL-6, IL-10, IL-8, IP-10, MCP-1, MCP-2, IL-1Ra, GRO-α, MIP-3α, TNF-RII, IL-7, MMP-9, CRP, SAA, MMP-3, MDC, YKL-40, IL-27, or a combination of two or more thereof. In embodiments, “immunostimulatory” refers the ability of the anti-CD73 compounds described herein to increase activation markers in a patient. In embodiments, the activation markers are B cells, dendritic cells, CD69, CD83, CD25, or a combination of two or more thereof. In embodiments, the activation marker is a B cell. In embodiments, the activation marker is a dendritic cell. In embodiments, the activation marker is CD69. In embodiments, the activation marker is CD83. In embodiments, the activation marker is CD25. In embodiments, the activation marker in the biological sample is increased relative to a control (e.g., relative to the activation markers in a biological sample taken prior to administration of an anti-CD73 antibody). In embodiments, “immunostimulatory” refers to the ability of the anti-CD73 antibody compound to increase antigen presenting cells in a subject. In embodiments, “immunostimulatory” refers to the ability of the anti-CD73 antibody compound to increase B cells in the subject. In embodiments, “immunostimulatory” refers to the ability of the anti-CD73 antibody compound to increase dendritic cells in the subject. In embodiments, “immunostimulatory” refers to the ability of the anti-CD73 antibody compound to increase antigen presenting cells in the subject, wherein the antigen-presenting cells express (i.e., comprise) CD3, CD14, CD19, CD25, CD69, CD83, CD86, MHC Class II (e.g., HLA-DR), BDCA-2, BDCA-4, CD11c^{low}, CD45RA, CD123, ILT-7, TLR7, TLR9, or a combination of two or more thereof. In embodiments. “immunostimulatory” refers to the ability of the anti-CD73 antibody compound to increase the CD4/CD8 ratio in the subject.

“Lymphoid cell” is used in accordance with its plain and ordinary meaning and refers to a subset of white blood cells responsible for immunity, and include T cells, B cells, and natural killer cells. T cells and B cells form cellular components of the adaptive immune response upon recognition of antigens. Natural killer cells are part of the innate immune system and defend the host from cancer cells and virally infected cells, for example through recognition of changes in cell surface receptors such as MHC class I.

“Dendritic cell” is used in accordance with its plain and ordinary meaning and refers to an antigen-presenting cell of the immune system. Dendritic cells process and present antigens on their surfaces for recognition by T cells.

“Plasmacytoid dendritic cell” (pDC) refers to a type of immune cell that links the innate and adaptive immune systems and may participate in antiviral mechanisms. For example, pDCs secrete large quantities of type 1 interferon (IFNs) in response to a viral infection. In contrast to conventional dendritic cells that leave the bone marrow as precursors, pDCs leave the bone marrow upon completion of development and go to lymphoid organs and peripheral blood.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

For specific proteins (antibodies or fragments thereof) described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI sequence reference. In embodiments, the protein is the protein as identified by its NCBI sequence reference, homolog or functional fragment thereof.

An amino acid residue in a protein “corresponds” to a given residue when it occupies the same essential structural position within the protein as the given residue.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. n the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain.

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutyric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]-hept-5-ene-2-carboxylic acid hydrochloride, cis-2-aminocycloheptane-carboxylic acid hydrochloride, cis-6-amino-3-cyclohexene-1-carboxylic acid hydrochloride, cis-2-amino-2-methylcyclohexanecarboxylic acid hydrochloride, cis-2-amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)-OH, Boc-Phe(4-Br)-OH, Boc-D-Phe(4-Br)-OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)-acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]-acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-j-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)-OH, Fmoc-Phe(4-Br)-OH, Fmoc-Phe(3,5-F2)-OH, Fmoc-β-(4-thiazolyl)-Ala-OH, Fmoc-3-(2-thienyl)-Ala-OH, and 4-(hydroxymethyl)-D-phenylalanine.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., http://www.ncbi.nlm.nih.gov/BLAST/ or the like). Such sequences are then the to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “antigen” as provided herein refers to molecules capable of binding to the antibody binding domain provided herein. An “antigen binding domain” as provided herein is a region of an antibody that binds to an antigen (epitope). As described above, the antigen binding domain may include one constant and one variable domain of each of the heavy and the light chain (VL, VH, CL and CH1, respectively). In embodiments, the antigen binding domain includes a light chain variable domain and a heavy chain variable domain. In embodiments, the antigen binding domain includes light chain variable domain and does not include a heavy chain variable domain and/or a heavy chain constant domain. The paratope or antigen-binding site is formed on the N-terminus of the antigen binding domain. The two variable domains of an antigen binding domain may bind the epitope of an antigen.

The term “viral antigen” refers to a viral substance that is capable of inducing an immune response in a subject. Viral antigens include a live virus, an inactivated virus, a toxoid produced by a virus, a viral vector, a viral subunit (e.g., protein or polysaccharide derived from the virus), or a nucleic acid (DNA or RNA).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv, or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (e.g., Fundamental Immunology (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al., WO91/17271; McCafferty et al., WO92/001047; Winter, WO92/20791; and Winter, FEBS Lett. 23:92, 1998) or using transgenic animals (Lonberg et al., WO93/12227; Kucherlapati WO91/10741).

Other approaches to design humanized antibodies may also be used to achieve the same result as the methods in U.S. Pat. Nos. 5,530,101 and 5,585,089 described above, for example, “superhumanization” (see Tan et al. J. Immunol. 169: 1119, 2002, and U.S. Pat. No. 6,881,557) or the method of Studnicak et al., Protein Eng. 7:805, 1994. Moreover, other approaches to produce genetically engineered, reduced-immunogenicity mAbs include reshaping, hyperchimerization and veneering/resurfacing, as described, e.g., in Vaswami et al., Annals of Allergy, Asthma and Immunology 81:105, 1998; Roguska et al. Protein Eng. 9:895, 1996; and U.S. Pat. Nos. 6,072,035 and 5,639,641.

A humanized antibody is a genetically engineered antibody in which at least one CDR (or functional fragment thereof) from a mouse antibody (“donor antibody” which can also be rat, hamster or other non-human species) are grafted onto a human antibody (“acceptor antibody”). The human antibody is a non-natural (e.g. not naturally occurring or not naturally produced by a human) antibody that does not elicit an immune response in a human, does not elicit a significant immune response in a human, or elicits an immune response that is less than the immune response elicited in a mouse. In embodiments, more than one mouse CDR is grafted (e.g. all six mouse CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, a humanized antibody may be an antibody having one or more CDRs from a donor antibody and a variable region framework (FR). The FR may form part of a constant region and/or a variable region within a human antibody. In addition, in order to retain high binding affinity, amino acids in the human acceptor sequence may be replaced by the corresponding amino acids from the donor sequence, for example where: (1) the amino acid is in a CDR; (2) the amino acid is in the human framework region (e.g. the amino acid is immediately adjacent to one of the CDR's). See, U.S. Pat. Nos. 5,530,101 and 5,585,089, which provide detailed instructions for construction of humanized antibodies. Although humanized antibodies often incorporate all six CDRs (e.g. as defined by Kabat, but often also including hypervariable loop H1 as defined by Chothia) from a mouse antibody, they can also be made with fewer mouse CDRs and/or less than the complete mouse CDR sequence (e.g. a functional fragment of a CDR) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

Typically a humanized antibody as provided herein may include (i) a light chain comprising at least one CDR (often three CDRs) from a mouse antibody (also referred to herein as a mouse CDR) and a human variable region framework; and (ii) a heavy chain comprising at least one CDR (often three CDRs) from the mouse antibody and a human variable region framework (FR). The light and heavy chain variable region frameworks (FRs) may each be a mature human antibody variable region framework sequence (or fragment thereof), a germline variable region framework sequence (combined with a J region sequence) (or fragment thereof), or a consensus sequence of a human antibody variable region framework sequence (or fragment thereof). In embodiments, the humanized antibody includes a light chain as described in (i), a heavy chain as described in (ii) together with a light chain human constant region and a heavy chain constant region.

A “CD73 protein” or “CD73 antigen” as referred to herein includes any of the recombinant or naturally-occurring forms of the Cluster of Differentiation 73 (CD73) also known as 5′-nucleotidase (5′-NT) or ecto-5′-nucleotidase or variants or homologs thereof that maintain CD73 nucleotidase activity (e.g. within at least 50%, 80%, 90%, 95%, 96%, 97%, 98%, 99% or 100% activity compared to CD73). In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD73 protein. In embodiments, the CD73 protein is substantially identical to the protein identified by the UniProt reference number 21589 or a variant or homolog having substantial identity thereto. In embodiments, the CD73 protein is substantially identical to the protein identified by the UniProt reference number Q61503 or a variant or homolog having substantial identity thereto.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Lymphoid cell” is used in accordance with its plain ordinary meaning and refers to a subset of white blood cells responsible for immunity, and include T cells, B cells, and natural killer cells. T cells and B cells form cellular components of the adaptive immune response upon recognition of antigens. Natural killer cells are part of the innate immune system and defend the host from cancer cells and virally infected cells, for example through recognition of changes in cell surface receptors such as MHC class I.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In embodiments, a control is the measurement of the activity of a protein in the absence of a compound as described herein (including embodiments and examples).

A “standard control” as provided herein refers to a sample that serves as a reference, usually a known reference, for comparison to a test sample. For example, a test sample can be taken from a patient suspected of having a disease (e.g., cancer) and compared to samples from a patient known to have the disease, or a known normal (non-disease) individual. A control can also represent an average value gathered from a population of similar individuals, e.g., disease patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In embodiments, contacting includes allowing a compound described herein to interact with a protein or enzyme that is involved in a signaling pathway.

As defined herein, the term “activation”, “activate”, “activating”, “activator” and the like in reference to a cell (e.g., B cell)-ligand interaction means positively affecting (e.g. increasing) the activity or function of the cell relative to the activity or function of the cell in the absence of the ligand. In embodiments activation means positively affecting (e.g. increasing) the proliferation rate or biologic activity of the cell relative to the rate or activity of the cell in the absence of the activator. The terms may reference activation, or activating, sensitizing, or up-regulating signal transduction or enzymatic activity or gene expression of a cell. Thus, activation may include, at least in part, partially or totally increasing stimulation, increasing or enabling activation, or activating, sensitizing, or up-regulating signal transduction or enzymatic activity or gene expression relative to the absence of the activator. Activation may include, at least in part, partially or totally increasing stimulation, increasing or enabling activation, or activating, sensitizing, or up-regulating signal transduction or enzymatic activity or gene expression.

The terms “agonist,” “activator,” “upregulator,” etc. refer to a substance capable of detectably increasing the activity or proliferation of a given cell. The agonist can increase activity or proliferation by 10%, 20%, 30%, 40%, 50%, 60%, 70%, 80%, 90% or more in comparison to a control in the absence of the agonist. In embodiments, proliferation or activity is 1.5-fold, 2-fold, 3-fold, 4-fold, 5-fold, 10-fold or higher than the proliferation or activity in the absence of the agonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to an agent that increases or decreases the level of a target molecule or the function of a target molecule or the physical state of the target of the molecule relative to the absence of the modulator. In embodiments, the modulator increases or decreases the proliferation rate of a cell (e.g., B cell) or the function of a cell or the physical state of a cell relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The terms “disease” or “condition” refer to a state of being or health status of a patient or subject capable of being treated with the compounds or methods provided herein. The disease may be a cancer, i.e., oncovirus-positive cancer. In embodiments, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin lymphoma (e.g., Burkitt's, small cell, and large cell lymphomas), Hodgkin lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin disease, and Non-Hodgkin lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “head and neck cancer” refers broadly to any cancer of the head and neck. In embodiments, the term “nead and neck cancer” refers to cancers in the larynx, throat (pharynx), lips, mouth, nose, and salivary glands. In embodiments, the head and neck cancer is laryngeal cancer, hypopharyngeal cancer, oral cavity cancer, squamous cell cancer of the lymph nodes in the neck, nasopharyngeal cancer, oropharyngeal cancer, paranasal sinus cancer, nasal cavity cancer, salivary gland cancer, and the like. Oral cavity refers to the outside and inside of the mouth, including lips, gingiva, hard palate, soft palate, retromolar trigone, tongue (front two-thirds), uvula, tonsil, buccal mucosa (lip and cheek lining), and the floor of the mouth. Oropharynx includes the soft palate, side and back wall of the throat, tonsils, and back third of the tongue.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin disease. Hodgkin disease represents approximately 15% of all diagnosed lymphomas. This is a cancer associated with Reed-Sternberg malignant B lymphocytes. Non-Hodgkin lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, mvxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration is required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound (antibody or other agent) described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

The term “CD4” as referred to herein is a glycoprotein expressed on the surface of T helper cells, regulatory T cells, monocytes, macrophages, and dendritic cells. CD4 was originally known as leu-3 and T4 (after the OKT4 monoclonal antibody). CD4 as referred to herein has four immunoglobulin domains (D₁ to D₄) that are exposed on the extracellular surface of the cell, see ENTREZ No. 920, UNIPROT No. P01730, and GENBANK@ Accession No. NP-000607, which are incorporated by reference.

The term “CD8” as referred to herein is a transnembrane glycoprotein that serves as a co-receptor for the T cell receptor (TCR) Like the TCR, CD8 binds to a major histocompatibility complex (MHC) molecule, but is specific for the class I MHC protein, see ENTREZ No. 925 and UNIPROT No. P01732, which are incorporated by reference herein.

The term “CD19 protein” or “CD19” as used herein includes any of the recombinant or naturally-occurring forms of B-lymphocyte antigen CD19, also known as CD19 molecule (Cluster of Differentiation 19), B-Lymphocyte Surface Antigen B4, T-Cell Surface Antigen Leu-12 and CVID3, or variants or homologs thereof that maintain CD19 activity (e.g. within at least 50%, 80%, 90%, 95%, 96%, 97%, 98%, 99% or 100% activity compared to CD19). In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD19 protein. In embodiments, the CD19 protein is substantially identical to the protein identified by the UniProt reference number P15391 or a variant or homolog having substantial identity thereto.

“CD73 levels” as referred to herein is the level of CD73 expressed by a tumor.

“An elevated level of CD73” as referred to herein is an elevated level of CD73 expressed (e.g., mRNA, proteins) by a tumor in a subject when compared to a control. CD73 levels can be measured from biological samples, such as a tumor sample (e.g., resected, biopsy) or a blood sample (e.g., peripheral blood), obtained from a subject. A tumor can be a primary tumor or a metastasis. A tumor as provided herein is a cellular mass including cancer cells and non-cancer cells. The non-cancer cells forming part of a tumor may be stromal cells, and immune cells (e.g., T cells, dendritic cells, B cells, macrophages). Thus, the elevated level of CD73 may be expressed by a non-cancer cell (e.g., a stromal cell) or a cancer cell (e.g., a malignant T cell). The term is further defined herein.

“Biological sample” refers to any biological sample taken from a subject. Biological samples include blood, plasma, serum, tumors, tissue, cells, and the like. In embodiments, the biological sample is a blood sample. In embodiments, the biological sample is a peripheral blood sample. In embodiments, the biological sample is a tumor sample. In embodiments, the biological sample is a primary tumor sample. In embodiments, the biological sample is a metastatic tumor sample. In embodiments, the biological sample is a resected tumor sample. In embodiments, the biological sample is a tumor biopsy sample. In embodiments, the biological sample is a resected tumor sample from a primary tumor. In embodiments, the biological sample is a resected tumor sample from a metastisic tumor. In embodiments, the biological sample is a tumor biopsy sample from a primary tumor. In embodiments, the biological sample is a tumor biopsy sample from a metastisic tumor. Biological samples can be taken from a subject by methods known in the art, and can be analyzed by methods known in the art.

### Anti-CD73 Antibodies

Any CD-73 compound can be used in the methods described herein. In embodiments, the anti-CD73 compound is an anti-CD73 antibody. In embodiments, the anti-CD73 antibodies used for the methods described herein, including embodiments thereof, are immunostimulatory. Any of the anti-CD73 antibodies (e.g., 1E9 antibodies) described in WO 2017/100670 may be used for the methods described herein. In embodiments, the anti-CD73 antibody is CPI-006. In embodiments, the anti-CD73 antibody is AD2. In embodiments, the anti-CD73 antibody is oleclumab. In embodiments, the anti-CD73 antibody is uliledlimab.

The anti-CD73 antibodies as provided herein are capable of binding a CD73 protein, activate and redistribute B cells and include the CDRs (CDR L1, CDR L2, CDR L3, CDR H1, CDR H2, and CDR 113) or functional fragments thereof of the mouse monoclonal antibody 1E9 (also referred to herein as the 1E9 antibody) (Thomson L F et al. Tissue Antigens 2008, Volume 35, Issue 1). The antibodies described herein including embodiments thereof maybe used for any of the methods or compositions described herein and below.

In embodiments, the anti-CD73 antibody comprises a humanized light chain variable region including an 1E9 antibody CDR L1, an 1E9 antibody CDR L2, and an 1E9 antibody CDR L3 and a humanized heavy chain variable region including an 1E9 antibody CDR H1, an 1E9 antibody CDR H2, and an 1E9 antibody CDR H3. In embodiments, the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and the CDR H3 has a sequence of SEQ ID NO:6. In embodiments, the humanized light chain variable region includes at least one binding framework region residue. In embodiments, the humanized heavy chain variable region includes at least one binding framework region residue. A framework region residue involved in (or important for) epitope binding (e.g. CD73 binding) is referred to herein as a binding framework region residue. The binding framework region residues may reside in the framework region of a humanized light chain variable region (i.e. FR L1, FR L2, FR L3, FR L4) or they may reside in the framework of a humanized heavy chain variable region (i.e. FR H1, FR H2, FR H3, FR 114). A binding framework residue residing in the FR L3 region of a humanized light chain is referred to herein as a FR L3 binding framework region residue. Thus, a binding framework region residue residing in the FR H3 region of a humanized heavy chain is referred to herein as a FR H3 binding framework region residue.

In embodiments, the anti-CD73 antibody comprises a humanized light chain variable region and a humanized heavy chain variable region. The humanized light chain variable region may include: (i) a CDR L1 as set forth in SEQ ID NO:1, a CDR L2 as set forth in SEQ ID NO:2, a CDR L3 as set forth in SEQ ID NO:3 and (ii) a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87.

In embodiments, the humanized heavy chain variable region comprises: (i) a mouse CDR H1 as set forth in SEQ ID NO:4, a mouse CDR H2 as set forth in SEQ ID NO:5, and a mouse CDR H3 as set forth in SEQ ID NO:6 and (ii) an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized light chain variable region comprises a binding framework region residue that is a valine at a position corresponding to Kabat position 2, a methionine at a position corresponding to Kabat position 4, an aspartic acid or a leucine at a position corresponding to Kabat position 9, a proline or a serine at a position corresponding to Kabat position 12, a lysine or a proline at a position corresponding to Kabat position 18, a alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100, a valine at a position corresponding to Kabat position 104, a glutamic acid or an alanine at a position corresponding to Kabat position 1, a glutamine at a position corresponding to Kabat position 3, a phenylalanine or a threonine at a position corresponding to Kabat position 10, a glutamine at a position corresponding to Kabat position 11, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a threonine at a position corresponding to Kabat position 22, a lysine at a position corresponding to Kabat position 42, an arginine at a position corresponding to Kabat position 45, an isoleucine at a position corresponding to Kabat position 58, a tyrosine at a position corresponding to Kabat position 67, a phenylalanine at a position corresponding to Kabat position 73, a tyrosine at a position corresponding to Kabat position 85, or a phenylalanine at a position corresponding to Kabat position 87.

In embodiments, the humanized light chain variable region comprises a binding framework region residue that is a valine at a position corresponding to Kabat position 2. In embodiments, the humanized light chain variable region includes a binding framework region residue that is a methionine at a position corresponding to Kabat position 4. In embodiments, the humanized light chain variable region includes a binding framework region residue that is an aspartic acid or a leucine at a position corresponding to Kabat position 9. In embodiments, the humanized light chain variable region includes a binding framework region residue that is a proline or a serine at a position corresponding to Kabat position 12. In embodiments, the humanized light chain variable region includes a binding framework region residue that is a lysine or a proline at a position corresponding to Kabat position 18. In embodiments, the humanized light chain variable region includes a binding framework region residue that is a alanine at a position corresponding to Kabat position 43. In embodiments, the humanized light chain variable region includes a binding framework region residue that is a proline or a serine at a position corresponding to Kabat position 60.

In embodiments, the humanized light chain variable region comprises a binding framework region residue that is a threonine at a position corresponding to Kabat position 74. In embodiments, the humanized light chain variable region includes a binding framework region residue that is an asparagine or a serine at a position corresponding to Kabat position 76. In embodiments, the humanized light chain variable region includes a binding framework region residue that is an asparagine or a serine at a position corresponding to Kabat position 77. In embodiments, the humanized light chain variable region includes a binding framework region residue that is an isoleucine or a leucine at a position corresponding to Kabat position 78. In embodiments, the humanized light chain variable region includes a binding framework region residue that is a serine or an alanine at a position corresponding to Kabat position 80. In embodiments, the humanized light chain variable region includes a binding framework region residue that is a glutamine at a position corresponding to Kabat position 100. In embodiments, the humanized light chain variable region includes a binding framework region residue that is a valine at a position corresponding to Kabat position 104. In embodiments, the humanized light chain variable region includes a binding framework region residue that is a glutamic acid or an alanine at a position corresponding to Kabat position 1. In embodiments, the humanized light chain variable region includes a binding framework region residue that is a glutamine at a position corresponding to Kabat position 3.

In embodiments, the humanized light chain variable region comprises a binding framework region residue that is a phenylalanine or a threonine at a position corresponding to Kabat position 10. In embodiments, the humanized light chain variable region includes a binding framework region residue that is a glutamine at a position corresponding to Kabat position 11. In embodiments, the humanized light chain variable region includes a binding framework region residue that is an alanine or a leucine at a position corresponding to Kabat position 13. In embodiments, the humanized light chain variable region includes a binding framework region residue that is a threonine at a position corresponding to Kabat position 14. In embodiments, the humanized light chain variable region includes a binding framework region residue that is a valine or a proline at a position corresponding to Kabat position 15. In embodiments, the humanized light chain variable region includes a binding framework region residue that is a lysine at a position corresponding to Kabat position 16. In embodiments, the humanized light chain variable region includes a binding framework region residue that is a glutamic acid or an aspartic acid at a position corresponding to Kabat position 17. In embodiments, the humanized light chain variable region includes a binding framework region residue that is a threonine at a position corresponding to Kabat position 22.

In embodiments, the humanized light chain variable region includes a binding framework region residue that is a lysine at a position corresponding to Kabat position 42. In embodiments, the humanized light chain variable region includes a binding framework region residue that is an arginine at a position corresponding to Kabat position 45. In embodiments, the humanized light chain variable region includes a binding framework region residue that is an isoleucine at a position corresponding to Kabat position 58. In embodiments, the humanized light chain variable region includes a binding framework region residue that is a tyrosine at a position corresponding to Kabat position 67. In embodiments, the humanized light chain variable region includes a binding framework region residue that is a phenylalanine at a position corresponding to Kabat position 73. In embodiments, the humanized light chain variable region includes a binding framework region residue that is an isoleucine at a position corresponding to Kabat position 78. In embodiments, the humanized light chain variable region includes a binding framework region residue that is a tyrosine at a position corresponding to Kabat position 85. In embodiments, the humanized light chain variable region includes a binding framework region residue that is a phenylalanine at a position corresponding to Kabat position 87.

In embodiments, the humanized heavy chain variable region comprises a binding framework region residue that is an isoleucine at a position corresponding to Kabat position 37, an alanine or a proline at a position corresponding to Kabat position 40, a lysine at a position corresponding to Kabat position 43, a serine at a position corresponding to Kabat position 70, an isoleucine or a threonine at a position corresponding to Kabat position 75, a tryptophan at a position corresponding to Kabat position 82, an arginine or a lysine at a position corresponding to Kabat position 83, a alanine at a position corresponding to Kabat position 84, a serine at a position corresponding to Kabat position 85, a valine or a methionine at a position corresponding to Kabat position 89, a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, a threonine at a position corresponding to Kabat position 87, a glutamic acid at a position corresponding to Kabat position 1, a valine at a position corresponding to Kabat position 24, a arginine at a position corresponding to Kabat position 44, a methionine at a position corresponding to Kabat position 48, a leucine at a position corresponding to Kabat position 80, or a glutamic acid at a position corresponding to Kabat position 81.

In embodiments, the humanized heavy chain variable region includes a binding framework region residue that is an isoleucine at a position corresponding to Kabat position 37. In embodiments, the humanized heavy chain variable region includes a binding framework region residue that is an alanine or a proline at a position corresponding to Kabat position 40. In embodiments, the humanized heavy chain variable region includes a binding framework region residue that is a lysine at a position corresponding to Kabat position 43. In embodiments, the humanized heavy chain variable region includes a binding framework region residue that is a serine at a position corresponding to Kabat position 70. In embodiments, the humanized heavy chain variable region includes a binding framework region residue that is an isoleucine or a threonine at a position corresponding to Kabat position 75. In embodiments, the humanized heavy chain variable region includes a binding framework region residue that is a tryptophan at a position corresponding to Kabat position 82. In embodiments, the humanized heavy chain variable region includes a binding framework region residue that is an arginine or a lysine at a position corresponding to Kabat position 83. In embodiments, the humanized heavy chain variable region includes a binding framework region residue that is a alanine at a position corresponding to Kabat position 84.

In embodiments, the humanized heavy chain variable region includes a binding framework region residue that is a serine at a position corresponding to Kabat position 85. In embodiments, the humanized heavy chain variable region includes a binding framework region residue that is a valine or a methionine at a position corresponding to Kabat position 89. In embodiments, the humanized heavy chain variable region includes a binding framework region residue that is a valine at a position corresponding to Kabat position 5. In embodiments, the humanized heavy chain variable region includes a binding framework region residue that is a serine at a position corresponding to Kabat position 7. In embodiments, the humanized heavy chain variable region includes a binding framework region residue that is a valine at a position corresponding to Kabat position 11. In embodiments, the humanized heavy chain variable region includes a binding framework region residue that is a glutamic acid or a lysine at a position corresponding to Kabat position 12. In embodiments, the humanized heavy chain variable region includes a binding framework region residue that is an isoleucine or a valine at a position corresponding to Kabat position 20. In embodiments, the humanized heavy chain variable region includes a binding framework region residue that is an arginine at a position corresponding to Kabat position 38. In embodiments, the humanized heavy chain variable region includes a binding framework region residue that is an arginine at a position corresponding to Kabat position 66. In embodiments, the humanized heavy chain variable region includes a binding framework region residue that is an valine at a position corresponding to Kabat position 67.

In embodiments, the humanized heavy chain variable region includes a binding framework region residue that is an isoleucine at a position corresponding to Kabat position 69. In embodiments, the humanized heavy chain variable region includes a binding framework region residue that is an alanine at a position corresponding to Kabat position 71. In embodiments, the humanized heavy chain variable region includes a binding framework region residue that is a lysine at a position corresponding to Kabat position 73. In embodiments, the humanized heavy chain variable region includes a binding framework region residue that is a threonine at a position corresponding to Kabat position 87. In embodiments, the humanized heavy chain variable region includes a binding framework region residue that is a glutamic acid at a position corresponding to Kabat position 1. In embodiments, the humanized heavy chain variable region includes a binding framework region residue that is a valine at a position corresponding to Kabat position 24. In embodiments, the humanized heavy chain variable region includes a binding framework region residue that is a arginine at a position corresponding to Kabat position 44. In embodiments, the humanized heavy chain variable region includes a binding framework region residue that is a methionine at a position corresponding to Kabat position 48. In embodiments, the humanized heavy chain variable region includes a binding framework region residue that is a leucine at a position corresponding to Kabat position 80. In embodiments, the humanized heavy chain variable region includes a binding framework region residue that is a glutamic acid at a position corresponding to Kabat position 81.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, and a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, or a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, or a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, and a methionine at a position corresponding to Kabat position 89.

In embodiments, the humanized light chain variable region includes a valine at a position corresponding to Kabat position 2, a methionine at a position corresponding to Kabat position 4, a leucine at a position corresponding to Kabat position 9, a proline at a position corresponding to Kabat position 12, and a proline at a position corresponding to Kabat position 18; and the humanized heavy chain variable region includes an isoleucine at a position corresponding to Kabat position 37, a proline at a position corresponding to Kabat position 40, a lysine at a position corresponding to Kabat position 43, a serine at a position corresponding to Kabat position 70, a isoleucine at a position corresponding to Kabat position 75, a tryptophan at a position corresponding to Kabat position 82, a lysine at a position corresponding to Kabat position 83, a alanine at a position corresponding to Kabat position 84, a serine at a position corresponding to Kabat position 85, and a methionine at a position corresponding to Kabat position 89.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 or a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 or a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, the humanized light chain variable region includes a proline or a serine at a position corresponding to Kabat position 12, an alanine at a position corresponding to Kabat position 43, a proline or a serine at a position corresponding to Kabat position 60, a threonine at a position corresponding to Kabat position 74, an asparagine or a serine at a position corresponding to Kabat position 76, an asparagine or a serine at a position corresponding to Kabat position 77, an isoleucine or a leucine at a position corresponding to Kabat position 78, a serine or an alanine at a position corresponding to Kabat position 80, a glutamine at a position corresponding to Kabat position 100 and a valine at a position corresponding to Kabat position 104; and the humanized heavy chain variable region includes a valine at a position corresponding to Kabat position 5, a serine at a position corresponding to Kabat position 7, a valine at a position corresponding to Kabat position 11, a glutamic acid or a lysine at a position corresponding to Kabat position 12, an isoleucine or a valine at a position corresponding to Kabat position 20, an arginine at a position corresponding to Kabat position 38, an alanine or a proline at a position corresponding to Kabat position 40, an arginine at a position corresponding to Kabat position 66, an valine at a position corresponding to Kabat position 67, an isoleucine at a position corresponding to Kabat position 69, an alanine at a position corresponding to Kabat position 71, an lysine at a position corresponding to Kabat position 73, an isoleucine or a threonine at a position corresponding to Kabat position 75, an arginine or a lysine at a position corresponding to Kabat position 83 and a threonine at a position corresponding to Kabat position 87.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 or a valine or a methionine at a position corresponding to Kabat position 89.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 or a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, humanized light chain variable region includes a glutamic acid or an alanine at a position corresponding to Kabat position 1, a valine at a position corresponding to Kabat position 2, a glutamine at a position corresponding to Kabat position 3, a methionine at a position corresponding to Kabat position 4, an aspartic acid or a leucine at a position corresponding to Kabat position 9, a phenylalanine or a threonine at a position corresponding to Kabat position 10, a glutamine at a position corresponding to Kabat position 11, a serine or a proline at a position corresponding to Kabat position 12, an alanine or a leucine at a position corresponding to Kabat position 13, a threonine at a position corresponding to Kabat position 14, a valine or a proline at a position corresponding to Kabat position 15, a lysine at a position corresponding to Kabat position 16, a glutamic acid or an aspartic acid at a position corresponding to Kabat position 17, a lysine or a proline at a position corresponding to Kabat position 18, a threonine at a position corresponding to Kabat position 22, a lysine at a position corresponding to Kabat position 42, an arginine at a position corresponding to Kabat position 45, an isoleucine at a position corresponding to Kabat position 58, a proline or a serine at a position corresponding to Kabat position 60, a tyrosine at a position corresponding to Kabat position 67, a phenylalanine at a position corresponding to Kabat position 73, an isoleucine at a position corresponding to Kabat position 78, a serine or an alanine at a position corresponding to Kabat position 80, a tyrosine at a position corresponding to Kabat position 85 and a phenylalanine at a position corresponding to Kabat position 87; and the humanized heavy chain variable region includes a glutamic acid at a position corresponding to Kabat position 1, a valine at a position corresponding to Kabat position 24, an isoleucine at a position corresponding to Kabat position 37, a lysine at a position corresponding to Kabat position 43, a arginine at a position corresponding to Kabat position 44, a methionine at a position corresponding to Kabat position 48, a serine at a position corresponding to Kabat position 70, a leucine at a position corresponding to Kabat position 80, a glutamic acid at a position corresponding to Kabat position 81, a tryptophan at a position corresponding to Kabat position 82, an alanine at a position corresponding to Kabat position 84, a serine at a position corresponding to Kabat position 85 and a valine or a methionine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, or a valine at a position corresponding to Kabat position 89.

In embodiments, the humanized heavy chain variable region includes a valine at a position corresponding to Kabat position 5, a serine at a position corresponding to Kabat position 7, a valine at a position corresponding to Kabat position 11, a glutamic acid at a position corresponding to Kabat position 12, a valine at a position corresponding to Kabat position 20, an arginine at a position corresponding to Kabat position 38, an alanine at a position corresponding to Kabat position 40, a methionine at a position corresponding to Kabat position 48, an arginine at a position corresponding to Kabat position 66, a valine at a position corresponding to Kabat position 67, an isoleucine at a position corresponding to Kabat position 69, an alanine at a position corresponding to Kabat position 71, a lysine at a position corresponding to Kabat position 73, a threonine at a position corresponding to Kabat position 75, a glutamic acid at a position corresponding to Kabat position 81, an arginine at a position corresponding to Kabat position 83, a threonine at a position corresponding to Kabat position 87, and a valine at a position corresponding to Kabat position 89.

In embodiments, the humanized heavy chain variable region includes the sequence of SEQ ID NO:7. In embodiments, the humanized heavy chain variable region is SEQ ID NO:7. In embodiments, the humanized light chain variable region includes the sequence of SEQ ID NO:8. In embodiments, the humanized light chain variable region is SEQ ID NO:8. In embodiments, the humanized heavy chain variable region includes the sequence of SEQ ID NO:7, and the humanized light chain variable region includes the sequence of SEQ ID NO:8. In embodiments, the humanized heavy chain variable region is SEQ ID NO:7, and the humanized light chain variable region is SEQ ID NO:8.

The anti-CD73 antibodies as provided herein may be Fab′ fragments. Where the anti-CD73 antibodies are Fab′ fragments, the anti-CD73 antibodies include a humanized heavy chain (e.g. including a constant and a variable region) and a humanized light chain (e.g. including a constant and a variable region). In embodiments, the anti-CD73 antibody is a Fab′ fragment. In embodiments, the anti-CD73 antibody includes a human constant region. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is an IgA. In embodiments, the anti-CD73 antibody is an IgM.

In embodiments, the anti-CD73 antibody is a single chain antibody. A single chain antibody includes a variable light chain and a variable heavy chain. A person of skill in the art will immediately recognize that a single chain antibody includes a single light chain and a single heavy chain, in contrast to an immunoglobulin antibody, which includes two identical pairs of polypeptide chains, each pair having one light chain and one heavy chain. Each light chain and heavy chain in turn consists of two regions: a variable (“V”) region (i.e. variable light chain and variable heavy chain) involved in binding the target antigen, and a constant (“C”) region that interacts with other components of the immune system. The variable light chain and the variable heavy chain in a single chain antibody may be linked through a linker peptide. Examples for linker peptides of single chain antibodies are described in Bird, R. E., Hardman, K. D., Jacobson, J. W., Johnson, S., Kaufman, B. M., Lee, S. M., Lee, T., Pope, S. H., Riordan, G. S. and Whitlow, M. (1988). Methods of making scFv antibodies have been described. See, Huse et al., Science 246:1275-1281 (1989); Ward et al., Nature 341:544-546 (1989); and Vaughan et al., Nature Biotech. 14:309-314 (1996). Briefly, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell.

The ability of an antibody to bind a specific epitope (e.g., CD73) can be described by the equilibrium dissociation constant (K_{D}). The equilibrium dissociation constant (K_{D}) as defined herein is the ratio of the dissociation rate (K-off) and the association rate (K-on) of an anti-CD73 antibody to a CD73 protein. It is described by the following formula: K_{D}=K-off/K-on. In embodiments, the anti-CD73 antibody is capable of binding a CD73 antigen with an equilibrium dissociation constant (K_{D}) from about 0.5 to about 25 nM. In embodiments, the anti-CD73 antibody is capable of binding a CD73 antigen with an equilibrium dissociation constant (K_{D}) from about 1 to about 25 nM. In embodiments, the anti-CD73 antibody is capable of binding a CD73 antigen with an equilibrium dissociation constant (K_{D}) from about 1.5 to about 25 nM. In embodiments, the anti-CD73 antibody is capable of binding a CD73 antigen with an equilibrium dissociation constant (K_{D}) from about 2 to about 25 nM. In embodiments, the anti-CD73 antibody is capable of binding a CD73 antigen with an equilibrium dissociation constant (K_{D}) from about 2.5 to about 25 nM. In embodiments, the anti-CD73 antibody is capable of binding a CD73 antigen with an equilibrium dissociation constant (K_{D}) from about 3 to about 25 nM. In embodiments, the anti-CD73 antibody is capable of binding a CD73 antigen with an equilibrium dissociation constant (K_{D}) from about 3.5 to about 25 nM. In embodiments, the anti-CD73 antibody is capable of binding a CD73 antigen with an equilibrium dissociation constant (K_{D}) from about 4 to about 25 nM. In embodiments, the anti-CD73 antibody is capable of binding a CD73 antigen with an equilibrium dissociation constant (K_{D}) in this paragraph at a pH below 7.5. In embodiments, the anti-CD73 antibody is capable of binding a CD73 antigen with an equilibrium dissociation constant (K_{D}) in this paragraph at a pH of less than about 7.5. In embodiments, the anti-CD73 antibody is capable of binding a CD73 antigen with an equilibrium dissociation constant (K_{D}) in this paragraph at a pH of less than about 7.0. In embodiments, the anti-CD73 antibody is capable of binding a CD73 antigen with an equilibrium dissociation constant (K_{D}) in this paragraph at a pH of less than about 6.5. In embodiments, the anti-CD73 antibody is capable of binding a CD73 antigen with an equilibrium dissociation constant (K_{D}) in this paragraph at a pH of less than about 6.0. In embodiments, the anti-CD73 antibody is capable of binding a CD73 antigen with an equilibrium dissociation constant (K_{D}) in this paragraph at a pH of less than about 5.5. In embodiments, the anti-CD73 antibody is capable of binding a CD73 antigen with an equilibrium dissociation constant (K_{D}) in this paragraph at a pH of less than about 5. In embodiments, the anti-CD73 antibody is capable of binding a CD73 antigen with an equilibrium dissociation constant (K_{D}) in this paragraph at a pH of less than about 4.5. In embodiments, the anti-CD73 antibody is capable of binding a CD73 antigen with an equilibrium dissociation constant (K_{D}) in this paragraph at a pH from about 6.0 to about 7.0. In embodiments, the anti-CD73 antibody is capable of binding a CD73 antigen with an equilibrium dissociation constant (K_{D}) in this paragraph at a pH of about 6.0. In embodiments, the anti-CD73 antibody is capable of binding a CD73 antigen with an equilibrium dissociation constant (K_{D}) in this paragraph at a pH of about 6.1. In embodiments, the anti-CD73 antibody is capable of binding a CD73 antigen with an equilibrium dissociation constant (K_{D}) in this paragraph at a pH of about 6.2. In embodiments, the anti-CD73 antibody is capable of binding a CD73 antigen with an equilibrium dissociation constant (K_{D}) in this paragraph at a pH of about 6.3. In embodiments, the anti-CD73 antibody is capable of binding a CD73 antigen with an equilibrium dissociation constant (K_{D}) in this paragraph at a pH of about 6.4. In embodiments, the anti-CD73 antibody is capable of binding a CD73 antigen with an equilibrium dissociation constant (K_{D}) in this paragraph at a pH of about 6.5. In embodiments, the anti-CD73 antibody is capable of binding a CD73 antigen with an equilibrium dissociation constant (K_{D}) in this paragraph at a pH of about 6.6. In embodiments, the anti-CD73 antibody is capable of binding a CD73 antigen with an equilibrium dissociation constant (K_{D}) in this paragraph at a pH of about 6.7. In embodiments, the anti-CD73 antibody is capable of binding a CD73 antigen with an equilibrium dissociation constant (K_{D}) in this paragraph at a pH of about 6.8. In embodiments, the anti-CD73 antibody is capable of binding a CD73 antigen with an equilibrium dissociation constant (K_{D}) in this paragraph at a pH of about 6.9. In embodiments, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) from about 4.5 to about 25 nM. In embodiments, the anti-CD73 antibody is capable of binding a CD73 antigen with an equilibrium dissociation constant (K_{D}) from about 5 to about 25 nM. In embodiments, the anti-CD73 antibody is capable of binding a CD73 antigen with an equilibrium dissociation constant (K_{D}) from about 5.5 to about 25 nM. In embodiments, the anti-CD73 antibody is capable of binding a CD73 antigen with an equilibrium dissociation constant (K_{D}) from about 6 to about 25 nM. In embodiments, the anti-CD73 antibody is capable of binding a CD73 antigen with an equilibrium dissociation constant (K_{D}) from about 6.5 to about 25 nM. In embodiments, the anti-CD73 antibody is capable of binding a CD73 antigen with an equilibrium dissociation constant (K_{D}) from about 7 to about 25 nM. In embodiments, the anti-CD73 antibody is capable of binding a CD73 antigen with an equilibrium dissociation constant (K_{D}) from about 7.5 to about 25 nM. In embodiments, the anti-CD73 antibody is capable of binding a CD73 antigen with an equilibrium dissociation constant (K_{D}) from about 8 to about 25 nM. In embodiments, the anti-CD73 antibody is capable of binding a CD73 antigen with an equilibrium dissociation constant (K_{D}) in this paragraph at a pH below 7.5. In embodiments, the anti-CD73 antibody is capable of binding a CD73 antigen with an equilibrium dissociation constant (K_{D}) in this paragraph at a pH of less than about 7.5. In embodiments, the anti-CD73 antibody is capable of binding a CD73 antigen with an equilibrium dissociation constant (K_{D}) in this paragraph at a pH of less than about 7.0. In embodiments, the anti-CD73 antibody is capable of binding a CD73 antigen with an equilibrium dissociation constant (K_{D}) in this paragraph at a pH of less than about 6.5. In embodiments, the anti-CD73 antibody is capable of binding a CD73 antigen with an equilibrium dissociation constant (K_{D}) in this paragraph at a pH of less than about 6.0. In embodiments, the anti-CD73 antibody is capable of binding a CD73 antigen with an equilibrium dissociation constant (K_{D}) in this paragraph at a pH of less than about 5.5. In embodiments, the anti-CD73 antibody is capable of binding a CD73 antigen with an equilibrium dissociation constant (K_{D}) in this paragraph at a pH of less than about 5. In embodiments, the anti-CD73 antibody is capable of binding a CD73 antigen with an equilibrium dissociation constant (K_{D}) in this paragraph at a pH of less than about 4.5. In embodiments, the anti-CD73 antibody is capable of binding a CD73 antigen with an equilibrium dissociation constant (K_{D}) in this paragraph at a pH from about 6.0 to about 7.0. In embodiments, the anti-CD73 antibody is capable of binding a CD73 antigen with an equilibrium dissociation constant (K_{D}) in this paragraph at a pH of about 6.0. In embodiments, the anti-CD73 antibody is capable of binding a CD73 antigen with an equilibrium dissociation constant (K_{D}) in this paragraph at a pH of about 6.1. In embodiments, the anti-CD73 antibody is capable of binding a CD73 antigen with an equilibrium dissociation constant (K_{D}) in this paragraph at a pH of about 6.2. In embodiments, the anti-CD73 antibody is capable of binding a CD73 antigen with an equilibrium dissociation constant (K_{D}) in this paragraph at a pH of about 6.3. In embodiments, the anti-CD73 antibody is capable of binding a CD73 antigen with an equilibrium dissociation constant (K_{D}) in this paragraph at a pH of about 6.4, In embodiments, the anti-CD73 antibody is capable of binding a CD73 antigen with an equilibrium dissociation constant (K_{D}) in this paragraph at a pH of about 6.5. In embodiments, the anti-CD73 antibody is capable of binding a CD73 antigen with an equilibrium dissociation constant (K_{D}) in this paragraph at a pH of about 6.6. In embodiments, the anti-CD73 antibody is capable of binding a CD73 antigen with an equilibrium dissociation constant (K_{D}) in this paragraph at a pH of about 6.7. In embodiments, the anti-CD73 antibody is capable of binding a CD73 antigen with an equilibrium dissociation constant (K_{D}) in this paragraph at a pH of about 6.8. In embodiments, the anti-CD73 antibody is capable of binding a CD73 antigen with an equilibrium dissociation constant (K_{D}) in this paragraph at a pH of about 6.9. In embodiments, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) from about 8.5 to about 25 nM. In embodiments, the anti-CD73 antibody is capable of binding a CD73 antigen with an equilibrium dissociation constant (K_{D}) from about 9 to about 25 nM. In embodiments, the anti-CD73 antibody is capable of binding a CD73 antigen with an equilibrium dissociation constant (K_{D}) from about 9.5 to about 25 nM. In embodiments, the anti-CD73 antibody is capable of binding a CD73 antigen with an equilibrium dissociation constant (K_{D}) from about 10 to about 25 nM. In embodiments, the anti-CD73 antibody is capable of binding a CD73 antigen with an equilibrium dissociation constant (K_{D}) from about 11 to about 25 nM. In embodiments, the anti-CD73 antibody is capable of binding a CD73 antigen with an equilibrium dissociation constant (K_{D}) from about 12 to about 25 nM. In embodiments, the anti-CD73 antibody is capable of binding a CD73 antigen with an equilibrium dissociation constant (K_{D}) from about 13 to about 25 nM. In embodiments, the anti-CD73 antibody is capable of binding a CD73 antigen with an equilibrium dissociation constant (K_{D}) from about 14 to about 25 nM. In embodiments, the anti-CD73 antibody is capable of binding a CD73 antigen with an equilibrium dissociation constant (K_{D}) from about 15 to about 25 nM. In embodiments, the anti-CD73 antibody is capable of binding a CD73 antigen with an equilibrium dissociation constant (K_{D}) from about 16 to about 25 nM. In embodiments, the anti-CD73 antibody is capable of binding a CD73 antigen with an equilibrium dissociation constant (K_{D}) in this paragraph at a pH below 7.5. In embodiments, the anti-CD73 antibody is capable of binding a CD73 antigen with an equilibrium dissociation constant (K_{D}) in this paragraph at a pH of less than about 7.5. In embodiments, the anti-CD73 antibody is capable of binding a CD73 antigen with an equilibrium dissociation constant (K_{D}) in this paragraph at a pH of less than about 7.0. In embodiments, the anti-CD73 antibody is capable of binding a CD73 antigen with an equilibrium dissociation constant (K_{D}) in this paragraph at a pH of less than about 6.5. In embodiments, the anti-CD73 antibody is capable of binding a CD73 antigen with an equilibrium dissociation constant (K_{D}) in this paragraph at a pH of less than about 6.0. In embodiments, the anti-CD73 antibody is capable of binding a CD73 antigen with an equilibrium dissociation constant (K_{D}) in this paragraph at a pH of less than about 5.5. In embodiments, the anti-CD73 antibody is capable of binding a CD73 antigen with an equilibrium dissociation constant (K_{D}) in this paragraph at a pH of less than about 5. In embodiments, the anti-CD73 antibody is capable of binding a CD73 antigen with an equilibrium dissociation constant (K_{D}) in this paragraph at a pH of less than about 4.5. In embodiments, the anti-CD73 antibody is capable of binding a CD73 antigen with an equilibrium dissociation constant (K_{D}) in this paragraph at a pH from about 6.0 to about 7.0. In embodiments, the anti-CD73 antibody is capable of binding a CD73 antigen with an equilibrium dissociation constant (K_{D}) in this paragraph at a pH of about 6.0. In embodiments, the anti-CD73 antibody is capable of binding a CD73 antigen with an equilibrium dissociation constant (K_{D}) in this paragraph at a pH of about 6.1. In embodiments, the anti-CD73 antibody is capable of binding a CD73 antigen with an equilibrium dissociation constant (K_{D}) in this paragraph at a pH of about 6.2. In embodiments, the anti-CD73 antibody is capable of binding a CD73 antigen with an equilibrium dissociation constant (K_{D}) in this paragraph at a pH of about 6.3. In embodiments, the anti-CD73 antibody is capable of binding a CD73 antigen with an equilibrium dissociation constant (K_{D}) in this paragraph at a pH of about 6.4. In embodiments, the anti-CD73 antibody is capable of binding a CD73 antigen with an equilibrium dissociation constant (K_{D}) in this paragraph at a pH of about 6.5. In embodiments, the anti-CD73 antibody is capable of binding a CD73 antigen with an equilibrium dissociation constant (K_{D}) in this paragraph at a pH of about 6.6. In embodiments, the anti-CD73 antibody is capable of binding a CD73 antigen with an equilibrium dissociation constant (K_{D}) in this paragraph at a pH of about 6.7. In embodiments, the anti-CD73 antibody is capable of binding a CD73 antigen with an equilibrium dissociation constant (K_{D}) in this paragraph at a pH of about 6.8. In embodiments, the anti-CD73 antibody is capable of binding a CD73 antigen with an equilibrium dissociation constant (K_{D}) in this paragraph at a pH of about 6.9. In embodiments, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) from about 17 to about 25 nM. In embodiments, the anti-CD73 antibody is capable of binding a CD73 antigen with an equilibrium dissociation constant (K_{D}) from about 18 to about 25 nM. In embodiments, the anti-CD73 antibody is capable of binding a CD73 antigen with an equilibrium dissociation constant (K_{D}) from about 19 to about 25 nM. In embodiments, the anti-CD73 antibody is capable of binding a CD73 antigen with an equilibrium dissociation constant (K_{D}) from about 20 to about 25 nM. In embodiments, the anti-CD73 antibody is capable of binding a CD73 antigen with an equilibrium dissociation constant (K_{D}) from about 21 to about 25 nM. In embodiments, the anti-CD73 antibody is capable of binding a CD73 antigen with an equilibrium dissociation constant (K_{D}) from about 22 to about 25 nM. In embodiments, the anti-CD73 antibody is capable of binding a CD73 antigen with an equilibrium dissociation constant (K_{D}) from about 23 to about 25 nM. In embodiments, the anti-CD73 antibody is capable of binding a CD73 antigen with an equilibrium dissociation constant (K_{D}) from about 24 to about 25 nM. In embodiments, the anti-CD73 antibody is capable of binding a CD73 antigen with an equilibrium dissociation constant (K_{D}) of about 0.5, 1 2, 3, 4, 5, 6, 7, 8, 9, 10, 11, 12, 13, 14, 15, 16, 17, 18, 19, 20, 21, 22, 23, 24, or 25 nM. In embodiments, the anti-CD73 antibody is capable of binding a CD73 antigen with an equilibrium dissociation constant (K_{D}) in this paragraph at a pH below 7.5. In embodiments, the anti-CD73 antibody is capable of binding a CD73 antigen with an equilibrium dissociation constant (K_{D}) in this paragraph at a pH of less than about 7.5. In embodiments, the anti-CD73 antibody is capable of binding a CD73 antigen with an equilibrium dissociation constant (K_{D}) in this paragraph at a pH of less than about 7.0. In embodiments, the anti-CD73 antibody is capable of binding a CD73 antigen with an equilibrium dissociation constant (K_{D}) in this paragraph at a pH of less than about 6.5. In embodiments, the anti-CD73 antibody is capable of binding a CD73 antigen with an equilibrium dissociation constant (K_{D}) in this paragraph at a pH of less than about 6.0. In embodiments, the anti-CD73 antibody is capable of binding a CD73 antigen with an equilibrium dissociation constant (K_{D}) in this paragraph at a pH of less than about 5.5. In embodiments, the anti-CD73 antibody is capable of binding a CD73 antigen with an equilibrium dissociation constant (K_{D}) in this paragraph at a pH of less than about 5. In embodiments, the anti-CD73 antibody is capable of binding a CD73 antigen with an equilibrium dissociation constant (K_{D}) in this paragraph at a pH of less than about 4.5. In embodiments, the anti-CD73 antibody is capable of binding a CD73 antigen with an equilibrium dissociation constant (K_{D}) in this paragraph at a pH from about 6.0 to about 7.0. In embodiments, the anti-CD73 antibody is capable of binding a CD73 antigen with an equilibrium dissociation constant (K_{D}) in this paragraph at a pH of about 6.0. In embodiments, the anti-CD73 antibody is capable of binding a CD73 antigen with an equilibrium dissociation constant (K_{D}) in this paragraph at a pH of about 6.1. In embodiments, the anti-CD73 antibody is capable of binding a CD73 antigen with an equilibrium dissociation constant (K_{D}) in this paragraph at a pH of about 6.2. In embodiments, the anti-CD73 antibody is capable of binding a CD73 antigen with an equilibrium dissociation constant (K_{D}) in this paragraph at a pH of about 6.3. In embodiments, the anti-CD73 antibody is capable of binding a CD73 antigen with an equilibrium dissociation constant (K_{D}) in this paragraph at a pH of about 6.4. In embodiments, the anti-CD73 antibody is capable of binding a CD73 antigen with an equilibrium dissociation constant (K_{D}) in this paragraph at a pH of about 6.5. In embodiments, the anti-CD73 antibody is capable of binding a CD73 antigen with an equilibrium dissociation constant (K_{D}) in this paragraph at a pH of about 6.6. In embodiments, the anti-CD73 antibody is capable of binding a CD73 antigen with an equilibrium dissociation constant (K_{D}) in this paragraph at a pH of about 6.7. In embodiments, the anti-CD73 antibody is capable of binding a CD73 antigen with an equilibrium dissociation constant (K_{D}) in this paragraph at a pH of about 6.8. In embodiments, the anti-CD73 antibody is capable of binding a CD73 antigen with an equilibrium dissociation constant (K_{D}) in this paragraph at a pH of about 6.9. In embodiments, the anti-CD73 antibody is capable of binding a CD73 antigen with an equilibrium dissociation constant (K_{D}) in this paragraph at a pH of about 7.0.

In embodiments, the anti-CD73 antibody is capable of binding a CD73 antigen with an equilibrium dissociation constant (K_{D}) of about 7.1 nM. In embodiments, the anti-CD73 antibody is capable of binding a CD73 antigen with an equilibrium dissociation constant (K_{D}) of about 6.9 nM. In embodiments, the anti-CD73 antibody is capable of binding a CD73 antigen with an equilibrium dissociation constant (K_{D}) of about 9.4 nM. In embodiments, the anti-CD73 antibody is capable of binding a CD73 antigen with an equilibrium dissociation constant (K_{D}) of about 19.5 nM. In embodiments, the anti-CD73 antibody is capable of binding a CD73 antigen with an equilibrium dissociation constant (K_{D}) of about 17.8 nM. In embodiments, the anti-CD73 antibody is capable of binding a CD73 antigen with an equilibrium dissociation constant (K_{D}) of about 15.9 nM. In embodiments, the anti-CD73 antibody is capable of binding a CD73 antigen with an equilibrium dissociation constant (K_{D}) in this paragraph at a pH below 7.5. In embodiments, the anti-CD73 antibody is capable of binding a CD73 antigen with an equilibrium dissociation constant (K_{D}) in this paragraph at a pH of less than about 7.5. In embodiments, the anti-CD73 antibody is capable of binding a CD73 antigen with an equilibrium dissociation constant (K_{D}) in this paragraph at a pH of less than about 7.0. In embodiments, the anti-CD73 antibody is capable of binding a CD73 antigen with an equilibrium dissociation constant (K_{D}) in this paragraph at a pH of less than about 6.5. In embodiments, the anti-CD73 antibody is capable of binding a CD73 antigen with an equilibrium dissociation constant (K_{D}) in this paragraph at a pH of less than about 6.0. In embodiments, the anti-CD73 antibody is capable of binding a CD73 antigen with an equilibrium dissociation constant (K_{D}) in this paragraph at a pH of less than about 5.5. In embodiments, the anti-CD73 antibody is capable of binding a CD73 antigen with an equilibrium dissociation constant (K_{D}) in this paragraph at a pH of less than about 5. In embodiments, the anti-CD73 antibody is capable of binding a CD73 antigen with an equilibrium dissociation constant (K_{D}) in this paragraph at a pH of less than about 4.5. In embodiments, the anti-CD73 antibody is capable of binding a CD73 antigen with an equilibrium dissociation constant (K_{D}) in this paragraph at a pH from about 6.0 to about 7.0. In embodiments, the anti-CD73 antibody is capable of binding a CD73 antigen with an equilibrium dissociation constant (K_{D}) in this paragraph at a pH of about 6.0. In embodiments, the anti-CD73 antibody is capable of binding a CD73 antigen with an equilibrium dissociation constant (K_{D}) in this paragraph at a pH of about 6.1. In embodiments, the anti-CD73 antibody is capable of binding a CD73 antigen with an equilibrium dissociation constant (K_{D}) in this paragraph at a pH of about 6.2. In embodiments, the anti-CD73 antibody is capable of binding a CD73 antigen with an equilibrium dissociation constant (K_{D}) in this paragraph at a pH of about 6.3. In embodiments, the anti-CD73 antibody is capable of binding a CD73 antigen with an equilibrium dissociation constant (K_{D}) in this paragraph at a pH of about 6.4. In embodiments, the anti-CD73 antibody is capable of binding a CD73 antigen with an equilibrium dissociation constant (K_{D}) in this paragraph at a pH of about 6.5. In embodiments, the anti-CD73 antibody is capable of binding a CD73 antigen with an equilibrium dissociation constant (K_{D}) in this paragraph at a pH of about 6.6. In embodiments, the anti-CD73 antibody is capable of binding a CD73 antigen with an equilibrium dissociation constant (K_{D}) in this paragraph at a pH of about 6.7. In embodiments, the anti-CD73 antibody is capable of binding a CD73 antigen with an equilibrium dissociation constant (K_{D}) in this paragraph at a pH of about 6.8. In embodiments, the anti-CD73 antibody is capable of binding a CD73 antigen with an equilibrium dissociation constant (K_{D}) in this paragraph at a pH of about 6.9. In embodiments, the anti-CD73 antibody is capable of binding a CD73 antigen with an equilibrium dissociation constant (K_{D}) in this paragraph at a pH of about 7.0.

### Methods of Treatment

The disclosure provides methods of treating oncovirus-positive cancers in a patient by administering to the patient an effective amount of an anti-CD73 compound, such as an anti-CD73 antibody. In embodiments, the disclosure provides methods of treating an oncovirus-positive cancer in a patient in need thereof by detecting an oncovirus in a cancer cell obtained from the patient; and administering to the patient an effective amount of an anti-CD73 compound. The anti-CD73 compound, such as anti-CD73 antibody, can be any described herein, including embodiments thereof. The anti-CD73 compounds, such as the anti-CD73 antibodies, are immunostimulatory. The oncovirus can be any known in the art, such as human papilloma virus, hepatitis B virus, hepatitis C virus, cytomegalovirus, Epstein-Barr virus, human immunodeficiency virus, human herpes virus-8, polyomavirus (e.g., merkel cell, John Cunningham, BK), adenovirus, human T-cell leukemia virus type (e.g., HTLV-1, HTLV-2), and the like. In embodiments, the oncovirus is human papilloma virus, hepatitis B virus, hepatitis C virus, cytomegalovirus, Epstein-Barr virus, human herpes virus-8, polyomavirus (e.g., merkel cell, John Cunningham, BK), adenovirus, human T-cell leukemia virus type (e.g., HTLV-1, HTLV-2) The oncovirus-positive cancer can be any cancer caused by an oncovirus. Methods of detecting an oncovirus in a cancer cell obtained from the patient can be accomplished by any method known in the art. In embodiments, the cancer cell is obtained from the tumor microenvironment in the patient. In embodiments, the cancer cell is obtained from a tumor in the patient. In embodiments, the cancer cell is obtained from a primary tumor in the patient. In embodiments, the cancer cell is obtained from a metastatic tumor in the patient.

Provided herein are methods of treating an oncovirus-positive cancer in a patient by administering to the patient an effective amount of an anti-CD73 compound, wherein the oncovirus is human papilloma virus (HPV). In embodiments, the disclosure provides methods of treating an oncovirus-positive cancer in a patient in need thereof by detecting an oncovirus in a cancer cell obtained from the patient, wherein the oncovirus is a human papilloma virus; and administering to the patient an effective amount of an anti-CD73 compound. In embodiments, the HPV is HPV-16, HPV-18, HPV-31, HPV-33, HPV-34, HPV-35, HPV-39. HPV-45, HPV-51, HPV-52, HPV-56, HPV-58, HPV-59, HPV-65. HPV-66, HPV-68, HPV-69 or a combination of two or more thereof. In embodiments, the HPV is HPV-16, HPV-18, HPV-31, or a combination of two or more thereof. In embodiments, the HPV is HPV-16, HPV-18, or a combination thereof. In embodiments, the HPV is HPV-16. In embodiments, the HPV is HPV-18. In embodiments, the HPV is HPV-31. In embodiments, the HPV is HPV-33. In embodiments, the HPV is HPV-34. In embodiments, the HPV is HPV-35. In embodiments, the HPV is HPV-39. In embodiments, the HPV is HPV-45. In embodiments, the HPV is HPV-51. In embodiments, the HPV is HPV-52. In embodiments, the HPV is HPV-55. In embodiments, the HPV is HPV-56. In embodiments, the HPV is HPV-58. In embodiments, the HPV is HPV-65. In embodiments, the HPV is HPV-66. In embodiments, the HPV is HPV-68. In embodiments, the HPV is HPV-69. In embodiments, the HPV-positive cancer is head and neck cancer (e.g., oropharyngeal cancer), cervical cancer, penile cancer, vaginal cancer, anal cancer, vulvar cancer, bladder cancer (e.g., urinary bladder cancer), or breast cancer (e.g., triple-negative breast cancer). In embodiments, the HPV is HPV-16, HPV-18, HPV-31, HPV-33, HPV-34, HPV-35, HPV-39, HPV-45, HPV-51, HPV-52, HPV-56, HPV-58, HPV-65, HPV-66, HPV-69, or a combination of two or more thereof, and the oncovirus-positive cancer is head and neck cancer (e.g., oropharyngeal cancer), cervical cancer, penile cancer, vaginal cancer, anal cancer, vulvar cancer, bladder cancer (e.g., urinary bladder cancer), or breast cancer. In embodiments, the HPV is HPV-16, HPV-18, HPV-31, or a combination of two or more thereof, and the oncovirus-positive cancer is head and neck cancer (e.g., oropharyngeal cancer), cervical cancer, penile cancer, vaginal cancer, anal cancer, vulvar cancer, bladder cancer (e.g., urinary bladder cancer), or breast cancer. In embodiments, the HPV is HPV-16 and/or HPV-18, and the oncovirus-positive cancer is head and neck cancer (e.g., oropharyngeal cancer), cervical cancer, penile cancer, vaginal cancer, anal cancer, vulvar cancer, bladder cancer (e.g., urinary bladder cancer), or breast cancer. In embodiments, the oncovirus is HPV-16 and/or HPV-18 and the oncovirus-positive cancer is cervical cancer. In embodiments, the oncovirus is HPV-16 and the oncovirus-positive cancer is cervical cancer. In embodiments, the oncovirus is HPV-18 and the oncovirus-positive cancer is cervical cancer. In embodiments, the oncovirus is HPV-16 and/or HPV-18 and the oncovirus-positive cancer is penile cancer. In embodiments, the oncovirus is HPV-16 and the oncovirus-positive cancer is penile cancer. In embodiments, the oncovirus is HPV-18 and the oncovirus-positive cancer is penile cancer. In embodiments, the oncovirus is HPV-16 and/or HPV-18 and the oncovirus-positive cancer is tonsillar cancer. In embodiments, the oncovirus is HPV-16 and the oncovirus-positive cancer is tonsillar cancer. In embodiments, the oncovirus is HPV-18 and the oncovirus-positive cancer is tonsillar cancer. In embodiments, the oncovirus is HPV-16 and/or HPV-18 and the oncovirus-positive cancer is oropharyngeal cancer. In embodiments, the oncovirus is HPV-16 and the oncovirus-positive cancer is oropharyngeal cancer. In embodiments, the oncovirus is HPV-18 and the oncovirus-positive cancer is oropharyngeal cancer. In embodiments, the oncovirus is HPV-16 and/or HPV-18 and the oncovirus-positive cancer is vaginal cancer. In embodiments, the oncovirus is HPV-16 and the oncovirus-positive cancer is vaginal cancer. In embodiments, the oncovirus is HPV-18 and the oncovirus-positive cancer is vaginal cancer. In embodiments, the oncovirus is HPV-16 and/or HPV-18 and the oncovirus-positive cancer is anal cancer. In embodiments, the oncovirus is HPV-16 and the oncovirus-positive cancer is anal cancer. In embodiments, the oncovirus is HPV-18 and the oncovirus-positive cancer is anal cancer. In embodiments, the HPV is HPV-16, HPV-18, HPV-31, or a combination of two or more thereof, and the oncovirus-positive cancer is vulvar cancer. In embodiments, the HPV is HPV-16 and/or HPV-18, and the oncovirus-positive cancer is vulvar cancer. In embodiments, the HPV is HPV-16 and the oncovirus-positive cancer is vulvar cancer. In embodiments, the HPV is HPV-18, and the oncovirus-positive cancer is vulvar cancer. In embodiments, the HPV is HPV-31, and the oncovirus-positive cancer is vulvar cancer. In embodiments, the oncovirus is HPV-16 and/or HPV-18 and the oncovirus-positive cancer is bladder cancer. In embodiments, the oncovirus is HPV-16 and the oncovirus-positive cancer is bladder cancer. In embodiments, the oncovirus is HPV-18 and the oncovirus-positive cancer is bladder cancer. In embodiments, the HPV is HPV-16, HPV-18, HPV-39, or a combination of two or more thereof, and the oncovirus-positive cancer is breast cancer. In embodiments, the HPV is HPV-16 and/or HPV-18 and the oncovirus-positive cancer is breast cancer. In embodiments, the HPV is HPV-16 and the oncovirus-positive cancer is breast cancer. In embodiments, the HPV is HPV-18 and the oncovirus-positive cancer is breast cancer. In embodiments, the HPV is HPV-39 and the oncovirus-positive cancer is breast cancer. In embodiments, the breast cancer is triple-negative breast cancer. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is hepatitis B virus. In embodiments, the disclosure provides methods of treating an oncovirus-positive cancer in a patient in need thereof by detecting an oncovirus in a cancer cell obtained from the patient, wherein the oncovirus is a hepatitis B virus; and administering to the patient an effective amount of an anti-CD73 compound. In embodiments, the oncovirus is hepatitis B virus, and the oncovirus positive cancer is liver cancer, stomach cancer, colorectal cancer, oral cavity cancer, pancreatic cancer, or lymphoma (e.g., non-Hodgkin lymphoma). In embodiments, the oncovirus is hepatitis B virus, and the oncovirus positive cancer is liver cancer. In embodiments, the oncovirus is hepatitis B virus, and the oncovirus positive cancer is stomach cancer. In embodiments, the oncovirus is hepatitis B virus, and the oncovirus positive cancer is colorectal cancer. In embodiments, the oncovirus is hepatitis B virus, and the oncovirus positive cancer is oral cavity cancer. In embodiments, the oncovirus is hepatitis B virus, and the oncovirus positive cancer is pancreatic cancer. In embodiments, the oncovirus is hepatitis B virus, and the oncovirus positive cancer is lymphoma. In embodiments, the oncovirus is hepatitis B virus, and the oncovirus positive cancer is non-Hodgkin lymphoma. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO: 1, the CDR L2 having a sequence of SEQ ID NO:2, the CDR L3 having a sequence of SEQ ID NO: 3; the CDR H1 having a sequence of SEQ ID NO:4, the CDR H2 having a sequence of SEQ ID NO: 5, and the CDR H3 having a sequence of SEQ ID NO: 6. In embodiments, the anti-CD73 antibody comprises a 1E9 antibody CDR L1 of SEQ ID NO: 1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is hepatitis C virus. In embodiments, the disclosure provides methods of treating an oncovirus-positive cancer in a patient in need thereof by detecting an oncovirus in a cancer cell obtained from the patient, wherein the oncovirus is a hepatitis C virus; and administering to the patient an effective amount of an anti-CD73 compound. In embodiments, the oncovirus positive cancer is liver cancer, lymphoma (e.g., non-Hodgkin lymphoma), or head and neck cancer. In embodiments, the oncovirus is hepatitis C virus, and the oncovirus positive cancer is liver cancer, lymphoma (e.g., non-Hodgkin lymphoma), or head and neck cancer. In embodiments, the oncovirus is hepatitis C virus, and the oncovirus positive cancer is liver cancer. In embodiments, the oncovirus is hepatitis C virus, and the oncovirus positive cancer is lymphoma. In embodiments, the oncovirus is hepatitis C virus, and the oncovirus positive cancer is non-Hodgkin lymphoma. In embodiments, the oncovirus is hepatitis C virus, and the oncovirus positive cancer is head and neck cancer. In embodiments, the head and neck cancer is oral cavity cancer, throat cancer, or laryngeal cancer. In embodiments, the oncovirus is hepatitis C virus, and the oncovirus positive cancer is oral cavity cancer. In embodiments, the oncovirus is hepatitis C virus, and the oncovirus positive cancer is throat cancer (e.g., upper throat). In embodiments, the oncovirus is hepatitis C virus, and the oncovirus positive cancer is laryngeal cancer. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is cytomegalovirus (CMV). In embodiments, the disclosure provides methods of treating an oncovirus-positive cancer in a patient in need thereof by detecting an oncovirus in a cancer cell obtained from the patient, wherein the oncovirus is a cytomegalovirus; and administering to the patient an effective amount of an anti-CD73 compound. In embodiments, the cytomegalovirus is human cytomegalovirus. In embodiments, the oncovirus positive cancer is colon cancer, glioma, prostate cancer, breast cancer, salivary gland cancer, or neuroblastoma. In embodiments, the oncovirus is cytomegalovirus and the oncovirus positive cancer is colon cancer. In embodiments, the oncovirus is cytomegalovirus and the oncovirus positive cancer is glioma. In embodiments, the oncovirus is cytomegalovirus and the oncovirus positive cancer is prostate cancer. In embodiments, the oncovirus is cytomegalovirus and the oncovirus positive cancer is inflammatory breast cancer. In embodiments, the oncovirus is cytomegalovirus and the oncovirus positive cancer is salivary gland cancer. In embodiments, the oncovirus is cytomegalovirus and the oncovirus positive cancer is neuroblastoma. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO: 1, the CDR L2 having a sequence of SEQ ID NO:2, the CDR L3 having a sequence of SEQ ID NO: 3; the CDR H1 having a sequence of SEQ ID NO:4, the CDR H2 having a sequence of SEQ ID NO: 5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is Epstein-Barr virus (EBV). In embodiments, the disclosure provides methods of treating an oncovirus-positive cancer in a patient in need thereof by detecting an oncovirus in a cancer cell obtained from the patient, wherein the oncovirus is Epstein-Barr virus; and administering to the patient an effective amount of an anti-CD73 compound. In embodiments, the oncovirus positive cancer is head and neck cancer, lymphoma, breast cancer, or stomach cancer. In embodiments, the oncovirus is Epstein-Barr virus, and the oncovirus positive cancer is head and neck cancer. In embodiments, the oncovirus is Epstein-Barr virus, and the oncovirus positive cancer is nasopharyngeal cancer. In embodiments, the oncovirus is Epstein-Barr virus, and the oncovirus positive cancer is lymphoma. In embodiments, the lymphoma is Burkitt lymphoma, Hodgkin lymphoma, non-Hodgkin lymphoma, or nasal type extranodal NK/T-cell lymphoma. In embodiments, the oncovirus is Epstein-Barr virus, and the oncovirus positive cancer is Burkitt lymphoma. In embodiments, the oncovirus is Epstein-Barr virus, and the oncovirus positive cancer is Hodgkin lymphoma. In embodiments, the oncovirus is Epstein-Barr virus, and the oncovirus positive cancer is non-Hodgkin lymphoma. In embodiments, the oncovirus is Epstein-Barr virus, and the oncovirus positive cancer is nasal type extranodal NK/T-cell lymphoma. In embodiments, the oncovirus is Epstein-Barr virus, and the oncovirus positive cancer is breast cancer. In embodiments, the oncovirus is Epstein-Barr virus, and the oncovirus positive cancer is stomach cancer. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6, In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is human immunodeficiency virus (HIV). In embodiments, the disclosure provides methods of treating an oncovirus-positive cancer in a patient in need thereof by detecting an oncovirus in a cancer cell obtained from the patient, wherein the oncovirus is human immunodeficiency virus; and administering to the patient an effective amount of an anti-CD73 compound. In embodiments, the oncovirus positive cancer is melanoma, lymphoma (e.g., Hodgkin lymphoma), anal cancer, lung cancer, liver cancer, testicular cancer, head and neck cancer (e.g., oral cavity cancer, throat cancer). In embodiments, the oncovirus is human immunodeficiency virus, and the oncovirus positive cancer is melanoma. In embodiments, the oncovirus is human immunodeficiency virus, and the oncovirus positive cancer is lymphoma. In embodiments, the oncovirus is human immunodeficiency virus, and the oncovirus positive cancer is Hodgkin lymphoma. In embodiments, the oncovirus is human immunodeficiency virus, and the oncovirus positive cancer is anal cancer. In embodiments, the oncovirus is human immunodeficiency virus, and the oncovirus positive cancer is lung cancer. In embodiments, the oncovirus is human immunodeficiency virus, and the oncovirus positive cancer is testicular cancer. In embodiments, the oncovirus is human immunodeficiency virus, and the oncovirus positive cancer is head and neck cancer. In embodiments, the oncovirus is human immunodeficiency virus, and the oncovirus positive cancer is oral cavity cancer. In embodiments, the oncovirus is human immunodeficiency virus, and the oncovirus positive cancer is throat cancer. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO: 1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO: 1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is human herpes virus 8 (HHV-8). In embodiments, the disclosure provides methods of treating an oncovirus-positive cancer in a patient in need thereof by detecting an oncovirus in a cancer cell obtained from the patient, wherein the oncovirus is human herpes virus 8; and administering to the patient an effective amount of an anti-CD73 compound. In embodiments, the oncovirus positive cancer is Kaposi's sarcoma or lymphoma. In embodiments, the oncovirus is human herpes virus 8, and the oncovirus positive cancer is Kaposi's sarcoma or lymphoma. In embodiments, the oncovirus is human herpes virus 8, and the oncovirus positive cancer is Kaposi's sarcoma. In embodiments, the oncovirus is human herpes virus 8, and the oncovirus positive cancer is lymphoma. In embodiments, the oncovirus is human herpes virus 8, and the oncovirus positive cancer is primary effusion lymphoma. In embodiments, the oncovirus is human herpes virus 8, and the oncovirus positive cancer is multicentric Castleman disease. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO: 7, and a humanized light chain variable region comprises the sequence of SEQ ID NO: 8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is merkel cell polyomavirus. In embodiments, the disclosure provides methods of treating an oncovirus-positive cancer in a patient in need thereof by detecting an oncovirus in a cancer cell obtained from the patient, wherein the oncovirus is merkel cell polyomavirus; and administering to the patient an effective amount of an anti-CD73 compound. In embodiments, the oncovirus positive cancer is merkel cell carcinoma. In embodiments, the oncovirus is merkel cell polyomavirus, and the oncovirus positive cancer is merkel cell carcinoma. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is John Cunningham polyomavirus. In embodiments, the disclosure provides methods of treating an oncovirus-positive cancer in a patient in need thereof by detecting an oncovirus in a cancer cell obtained from the patient, wherein the oncovirus is John Cunningham polyomavirus; and administering to the patient an effective amount of an anti-CD73 compound. In embodiments, the oncovirus positive cancer is gastrointestinal cancer. In embodiments, the oncovirus is John Cunningham polyomavirus, and the oncovirus positive cancer is gastrointestinal cancer. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO: 7, and a humanized light chain variable region comprises the sequence of SEQ ID NO: 8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is BK polyomavirus. In embodiments, the disclosure provides methods of treating an oncovirus-positive cancer in a patient in need thereof by detecting an oncovirus in a cancer cell obtained from the patient, wherein the oncovirus is BK polyomavirus; and administering to the patient an effective amount of an anti-CD73 compound. In embodiments, the oncovirus positive cancer is lung cancer, pancreatic cancer, liver cancer, urogenital tract cancer, head and neck cancer, rhabdomyosarcoma, Kaposi's sarcoma, brain cancer, or bladder cancer. In embodiments, the oncovirus is BK polyomavirus, and the oncovirus positive cancer is lung cancer, pancreatic cancer, liver cancer, urogenital tract cancer, head and neck cancer, rhabdomyosarcoma, Kaposi's sarcoma, brain cancer, or bladder cancer. In embodiments, the oncovirus is BK polyomavirus, and the oncovirus positive cancer is lung cancer. In embodiments, the oncovirus is BK polyomavirus, and the oncovirus positive cancer is pancreatic cancer. In embodiments, the oncovirus is BK polyomavirus, and the oncovirus positive cancer is liver cancer. In embodiments, the oncovirus is BK polyomavirus, and the oncovirus positive cancer is urogenital tract cancer. In embodiments, the oncovirus is BK polyomavirus, and the oncovirus positive cancer is head and neck cancer. In embodiments, the oncovirus is BK polyomavirus, and the oncovirus positive cancer is rhabdomyosarcoma. In embodiments, the oncovirus is BK polyomavirus, and the oncovirus positive cancer is Kaposi's sarcoma. In embodiments, the oncovirus is BK polyomavirus, and the oncovirus positive cancer is brain cancer. In embodiments, the oncovirus is BK polyomavirus, and the oncovirus positive cancer is bladder cancer. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is adenovirus. In embodiments, the disclosure provides methods of treating an oncovirus-positive cancer in a patient in need thereof by detecting an oncovirus in a cancer cell obtained from the patient, wherein the oncovirus is adenovirus; and administering to the patient an effective amount of an anti-CD73 compound. In embodiments, the oncovirus-positive cancer is brain cancer, gastrointestinal cancer, or lymphoma. In embodiments, the oncovirus is adenovirus, and the oncovirus-positive cancer is brain cancer. In embodiments, the brain cancer is glioblastoma, oligodendroglioma, or ependymomas. In embodiments, the oncovirus is adenovirus, and the oncovirus-positive cancer is glioblastoma. In embodiments, the oncovirus is adenovirus, and the oncovirus-positive cancer is oligodendroglioma. In embodiments, the oncovirus is adenovirus, and the oncovirus-positive cancer is ependymomas. In embodiments, the oncovirus is adenovirus, and the oncovirus-positive cancer is gastrointestinal cancer. In embodiments, the oncovirus is adenovirus, and the oncovirus-positive cancer is lymphoma. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO:1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is human T-cell leukemia virus type-1 (HTLV-1). In embodiments, the disclosure provides methods of treating an oncovirus-positive cancer in a patient in need thereof by detecting an oncovirus in a cancer cell obtained from the patient, wherein the oncovirus is human T-cell leukemia virus type-1; and administering to the patient an effective amount of an anti-CD73 compound. In embodiments, the oncovirus-positive cancer is adult T-cell leukemia/lymphoma. In embodiments, the oncovirus is human T-cell leukemia virus type-1, and the oncovirus-positive cancer is adult T-cell leukemia/lymphoma. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO:3; the CDR H1 having a sequence of SEQ ID NO:4, the CDR H2 having a sequence of SEQ ID NO:5, and the CDR H3 having a sequence of SEQ ID NO:6. In embodiments, the anti-CD73 antibody comprises a 1E9 antibody CDR L1 of SEQ ID NO: 1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

Provided herein are methods of treating an oncovirus-positive cancer in a patient by administering to the patient an effective amount of an anti-CD73 compound, wherein the oncovirus is human T-cell leukemia virus type-2 (HTLV-2). In embodiments, the disclosure provides methods of treating an oncovirus-positive cancer in a patient in need thereof by detecting an oncovirus in a cancer cell obtained from the patient, wherein the oncovirus is human T-cell leukemia virus type-2; and administering to the patient an effective amount of an anti-CD73 compound. In embodiments, the oncovirus-positive cancer is cutaneous T-cell lymphoma. In embodiments, the oncovirus is human T-cell leukemia virus type-2, and the oncovirus-positive cancer is cutaneous T-cell lymphoma. In embodiments, the anti-CD73 compound is an anti-CD73 antibody. In embodiments, the anti-CD73 compound, such as an anti-CD73 antibody, is immunostimulatory. In embodiments, the anti-CD73 antibody is any anti-CD73 antibody described herein, including all embodiments thereof. In embodiments, the anti-CD73 antibody is capable of binding a CD73 protein, is capable of inhibiting CD73 catalytic activity, and is capable of activating B cells. In embodiments, the anti-CD73 antibody is capable of activating B cells. In embodiments, the anti CD73 antibody is capable of binding to a B cell and inducing expression of activation markers. In embodiments, the anti CD73 antibody is capable of binding to a B cell and inducing expression of activation markers and maturation into plasma cells. In embodiments, the anti CD73 antibody is capable of binding to a B cell and inducing the secretion of antibodies specific to the oncovirus. In embodiments, the anti CD73 antibody is capable of binding to a B cell and inducing the generation of memory B cells. In embodiments, the anti CD73 antibody is capable of binding to a B cell and inducing expression of activation markers, is capable of binding to a B cell and inducing the secretion of antibodies specific to the oncovirus, and is capable of binding to a B cell and inducing the generation of memory B cells. In embodiments, the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3. In embodiments, the anti-CD73 antibody comprises CDR L1 have a sequence of SEQ ID NO:1, the CDR L2 having a sequence of SEQ ID NO:2, the CDR L3 having a sequence of SEQ ID NO: 3; the CDR H1 having a sequence of SEQ ID NO:4, the CDR H2 having a sequence of SEQ ID NO: 5, and the CDR H3 having a sequence of SEQ ID NO: 6. In embodiments, the anti-CD73 antibody comprises a 1E9 antibody CDR L1 of SEQ ID NO: 1, a 1E9 antibody CDR L2 of SEQ ID NO:2, a 1E9 antibody CDR L3 of SEQ ID NO:3, a 1E9 antibody CDR H1 of SEQ ID NO:4, a 1E9 antibody CDR H2 of SEQ ID NO:5, and a 1E9 antibody CDR H3 of SEQ ID NO:6. In embodiments, the anti-CD73 antibody comprises a humanized heavy chain variable region comprises the sequence of SEQ ID NO:7. In embodiments, the anti-CD73 antibody comprises a humanized light chain variable region comprises the sequence of SEQ ID NO:8. In embodiments, the anti-CD73 antibody comprises a humanized heavy chain variable region comprises the sequence of SEQ ID NO:7, and a humanized light chain variable region comprises the sequence of SEQ ID NO:8. In embodiments, the anti-CD73 antibody comprises a glutamine at a position corresponding to Kabat position 297. In embodiments, the anti-CD73 antibody is an IgG. In embodiments, the anti-CD73 antibody is an IgG1. In embodiments, the anti-CD73 antibody is an IgG4. In embodiments, the anti-CD73 antibody is a Fab′ fragment. In embodiments, the anti-CD73 antibody is a single chain antibody (scFv). In embodiments, the anti-CD73 antibody is capable of binding a CD73 antigen with an equilibrium dissociation constant (K_{D}) from about 0.3 to about 25 nM. In embodiments, the anti-CD73 antibody is capable of binding a CD73 antigen at a pH from about 6.0 to about 7.0. In embodiments, the anti-CD73 antibody is CPI-006.

### Pharmaceutical Compositions

The anti-CD73 compounds can be administered in the form of a pharmaceutical composition. Pharmaceutical compositions containing antibodies are well known in the art, and the anti-CD73 compounds, such as anti-CD73 antibodies, described herein can be formulated as a pharmaceutical composition using methods and pharmaceutically acceptable excipients known in the art.

For parenteral administration, the anti-CD73 compounds, such as anti-CD73 antibodies, described herein can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). The anti-CD73 compounds, such as anti-CD73 antibodies, described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein. Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

Pharmaceutical compositions include compositions wherein the active ingredient (e.g. described herein and compositions described herein, including embodiments or examples) is contained in a therapeutically effective amount, i.e., in an amount effective to achieve its intended purpose. The actual amount effective for a particular application will depend, inter alia, on the condition being treated and the desired result, e.g., modulating the activity of a target molecule, and/or reducing, eliminating, or slowing the progression of disease symptoms. Determination of a therapeutically effective amount of the antibodies described herein is well within the capabilities of those skilled in the art, especially in light of the detailed disclosure herein.

The compositions described herein can include a single agent or more than one agent. The compositions for administration will commonly include the anti-CD73 compounds, such as anti-CD73 antibodies, described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the subject's needs.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the anti-CD73 compounds, such as anti-CD73 antibodies, described herein in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Solvents (e.g., dimethyl sulfoxide) can be used for rapid penetration, delivering high concentrations of the active agents to a small area.

The compositions of the anti-CD73 compounds, such as anti-CD73 antibodies, described herein can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods and antibodies described herein. Adjustment and manipulation of established dosages (e.g., frequency and duration) are well within the ability of those skilled in the art.

For any composition (e.g., the anti-CD73 antibody provided) described herein, the therapeutically effective amount can be initially determined from cell culture assays. Target concentrations will be those concentrations of active compound(s) that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects.

In embodiments, the anti-CD73 inhibitor is administered to subjects in an effective amount to treat the disease in the subject. In embodiments, the effective amount is from about 0.1 mg/kg to about 10.0 mg/kg. In embodiments, the effective amount is from about 0.2 mg/kg to about 6 mg/kg. In embodiments, the effective amount is from about 0.25 mg/kg to about 5.5 mg/kg. In embodiments, the effective amount is from about 0.3 mg/kg to about 5.0 mg/kg. In embodiments, the effective amount is from about 0.25 mg/kg to about 0.35 mg/kg. In embodiments, the effective amount is 0.3 mg/kg. In embodiments, the effective amount is from about 0.5 mg/kg to about 1.5 mg/kg. In embodiments, the effective amount is 1.0 mg/kg. In embodiments, the effective amount is from about 2 mg/kg to about 4 mg/kg. In embodiments, the effective amount is 3.0 mg/kg. In embodiments, the effective amount is from about 4 mg/kg to about 6 mg/kg. In embodiments, the effective amount is 5.0 mg/kg.

In embodiments, the effective amount is about 1 mg/kg, 3 mg/kg, 6 mg/kg, 10 mg/kg, 30 mg/kg, 40 mg/kg, or 120 mg/kg. In embodiments, the effective amount is about 0.05 mg/kg, 0.1 mg/kg, 0.3 mg/kg, 1 mg/kg, 3 mg/kg, 6 mg/kg, 10 mg/kg, 30 mg/kg, 40 mg/kg, or 120 mg/kg. In embodiments, the effective amount is about 0.05 mg/kg. In embodiments, the effective amount is 0.05 mg/kg. In embodiments, the effective amount is about 0.1 mg/kg. In embodiments, the effective amount is 0.1 mg/kg. In embodiments, the effective amount is about 0.3 mg/kg. In embodiments, the effective amount is 0.3 mg/kg. In embodiments, the effective amount is about 1 mg/kg. In embodiments, the effective amount is 1 mg/kg. In embodiments, the effective amount is about 3 mg/kg. In embodiments, the effective amount is 3 mg/kg. In embodiments, the effective amount is about 6 mg/kg. In embodiments, the effective amount is 6 mg/kg. In embodiments, the effective amount is about 10 mg/kg. In embodiments, the effective amount is 10 mg/kg. In embodiments, the effective amount is about 12 mg/kg. In embodiments, the effective amount is 12 mg/kg. In embodiments, the effective amount is about 30 mg/kg. In embodiments, the effective amount is 30 mg/kg. In embodiments, the effective amount is about 40 mg/kg. In embodiments, the effective amount is 40 mg/kg. In embodiments, the effective amount is about 120 mg/kg. In embodiments, the effective amount is 120 mg/kg. In embodiments, the effective amount administered results in serum levels of the antibody of about 10 μg/ml.

In embodiments, the anti-CD73 antibody is administered to a subject in an effective amount of at least 1 mg/kg. In embodiments, the effective amount is at least 2 mg/kg. In embodiments, the effective amount is at least 2 mg/kg. In embodiments, the effective amount is at least 3 mg/kg. In embodiments, the effective amount is at least 4 mg/kg. In embodiments, the effective amount is at least 5 mg/kg. In embodiments, the effective amount is at least 6 mg/kg. In embodiments, the effective amount is at least 7 mg/kg. In embodiments, the effective amount is at least 8 mg/kg. In embodiments, the effective amount is at least 9 mg/kg. In embodiments, the effective amount is at least 10 mg/kg. In embodiments, the effective amount is at least 11 mg/kg. In embodiments, the effective amount is at least 12 mg/kg. In embodiments, the effective amount is at least 13 mg/kg. In embodiments, the effective amount is at least 14 mg/kg. In embodiments, the effective amount is at least 15 mg/kg. In embodiments, the effective amount is from about 1 mg/kg to about 100 mg/kg. In embodiments, the effective amount is from about 2 mg/kg to about 90 mg/kg. In embodiments, the effective amount is from about 3 mg/kg to about 80 mg/kg. In embodiments, the effective amount is from about 4 mg/kg to about 70 mg/kg. In embodiments, the effective amount is from about 5 mg/kg to about 60 mg/kg. In embodiments, the effective amount is from about 6 mg/kg to about 50 mg/kg. In embodiments, the effective amount is from about 4 mg/kg to about 25 mg/kg. In embodiments, the effective amount is from about 5 mg/kg to about 25 mg/kg. In embodiments, the effective amount is from about 6 mg/kg to about 25 mg/kg. In embodiments, the effective amount is from about 7 mg/kg to about 25 mg/kg. In embodiments, the effective amount is from about 8 mg/kg to about 25 mg/kg. In embodiments, the effective amount is from about 9 mg/kg to about 25 mg/kg. In embodiments, the effective amount is from about 10 mg/kg to about 25 mg/kg. In embodiments, the effective amount is from about 5 mg/kg to about 15 mg/kg. In embodiments, the effective amount is from about 6 mg/kg to about 12 mg/kg. In embodiments, the effective amount is about 4 mg/kg. In embodiments, the effective amount is about 5 mg/kg. In embodiments, the effective amount is about 6 mg/kg. In embodiments, the effective amount is about 7 mg/kg. In embodiments, the effective amount is about 8 mg/kg. In embodiments, the effective amount is about 9 mg/kg. In embodiments, the effective amount is about 10 mg/kg. In embodiments, the effective amount is about 11 mg/kg. In embodiments, the effective amount is about 12 mg/kg. In embodiments, the effective amount is about 13 mg/kg. In embodiments, the effective amount is about 14 mg/kg. In embodiments, the effective amount is about 15 mg/kg. In embodiments, the effective amount is about 16 mg/kg. In embodiments, the effective amount is about 17 mg/kg. In embodiments, the effective amount is about 18 mg/kg. In embodiments, the effective amount is about 19 mg/kg. In embodiments, the effective amount is about 20 mg/kg. In embodiments, the effective amount is about 21 mg/kg. In embodiments, the effective amount is about 22 mg/kg. In embodiments, the effective amount is about 23 mg/kg. In embodiments, the effective amount is about 24 mg/kg. In embodiments, the effective amount is about 25 mg/kg.

In embodiments, the anti-CD73 antibody is administered by parenteral injection. In embodiments, the injection is a bolus injection. In embodiments, the injection is an infusion (e.g., over the course of 5 minutes to 2 hours; or from about 30 minutes to about 90 minutes). In embodiments, the anti-CD73 antibody is administered once per week (i.e., once every 7 days), once every two weeks (e.g., once every 14 days), once every three weeks (e.g., once every 21 days), or once per month (e.g., once every 28 days).

In embodiments for the methods provided herein the anti-CD73 antibody may be administered at a half maximal effective concentration (EC₅₀) of at least 100 nM (e.g., 100, 105, 110, 115, 120, 125, 130, 135, 140, 145, 150, 155, 160, 165, 170, 175, 180, 185, 190, 195, 200, 210, 220, 230, 240, 250 nM). In embodiments, the anti-CD73 antibody is administered at a half maximal effective concentration (EC₅₀) of at least 100 nM. In embodiments, the antibody is administered at a half maximal effective concentration (EC₅₀) of 110 nM. In embodiments, the antibody is administered at a half maximal effective concentration (EC₅₀) of 115 nM. In embodiments, the antibody is administered at a half maximal effective concentration (EC₅₀) of 120 nM. In embodiments, the antibody is administered at a half maximal effective concentration (EC₅₀) of 125 nM. In embodiments, the antibody is administered at a half maximal effective concentration (EC₅₀) of 130 nM. In embodiments, the antibody is administered at a half maximal effective concentration (EC₅₀) of 135 nM. In embodiments, the antibody is administered at a half maximal effective concentration (EC₅₀) of 140 nM. In embodiments, the antibody is administered at a half maximal effective concentration (EC₅₀) of 145 nM. In embodiments, the antibody is administered at a half maximal effective concentration (EC₅₀) of 150 nM. In embodiments, the antibody is administered at a half maximal effective concentration (EC₅₀) of 155 nM. In embodiments, the antibody is administered at a half maximal effective concentration (EC₅₀) of 160 nM. In embodiments, the antibody is administered at a half maximal effective concentration (EC₅₀) of 165 nM. In embodiments, the antibody is administered at a half maximal effective concentration (EC₅₀) of 170 nM. In embodiments, the antibody is administered at a half maximal effective concentration (EC₅₀) of 175 nM. In embodiments, the antibody is administered at a half maximal effective concentration (EC₅₀) of 180 nM. In embodiments, the antibody is administered at a half maximal effective concentration (EC₅₀) of 185 nM. In embodiments, the antibody is administered at a half maximal effective concentration (EC₅₀) of 190 nM. In embodiments, the antibody is administered at a half maximal effective concentration (EC₅₀) of 195 nM. In embodiments, the antibody is administered at a half maximal effective concentration (EC₅₀) of 200 nM. In embodiments, the antibody is administered at a half maximal effective concentration (EC₅₀) of 210 nM. In embodiments, the antibody is administered at a half maximal effective concentration (EC₅₀) of 220 nM. In embodiments, the antibody is administered at a half maximal effective concentration (EC₅₀) of 230 nM. In embodiments, the antibody is administered at a half maximal effective concentration (EC₅₀) of 240 nM. In embodiments, the antibody is administered at a half maximal effective concentration (EC₅₀) of 250 nM.

In embodiments, the antibody is administered at an EC₅₀ of about 137 nM. In embodiments, the antibody is administered at an EC₅₀ of 137 nM. In embodiments, the antibody is administered at an EC₅₀ of about 189 nM. In embodiments, the antibody is administered at an EC₅₀ of 189 nM.

“Pharmaceutically acceptable excipient” and “pharmaceutically acceptable carrier” refer to a substance that aids the administration of an active agent to and absorption by a subject and can be included in pharmaceutical compositions without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like. One of skill in the art will recognize that other pharmaceutical excipients can be used in the pharmaceutical compositions described herein.

In embodiments, the pharmaceutically acceptable carrier is an immunological adjuvant. In some examples, the immunological adjuvant can include, but is not limited to, agonists of Toll-like Receptors (TLRs), agonists of the STING pathway, agonistic antibodies against CD40, OX40, CTLA4, PD1, or PD1-L, Freund's adjuvant, bryostatins and ligands for CD40, OX40, CD137, PD1, CTLA4 and any combinations thereof. In embodiments, the adjuvant can increase immunogenicity that is induced when a cell-penetrating complex by co-administered with the complex to a subject.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

### EMBODIMENTS

Embodiment 1. A method of treating an oncovirus positive cancer in a patient in need thereof, the method comprising administering to the patient an effective amount of an anti-CD73 compound.

Embodiment 2. A method of treating a cancer in a patient in need thereof, the method comprising: (i) detecting an oncovirus in a cancer cell obtained from the patient; and (ii) administering to the patient an effective amount of an anti-CD73 compound.

Embodiment 3. The method of Embodiment 1 or 2, wherein the oncovirus is human papilloma virus.

Embodiment 4. The method of Embodiment 3, wherein the human papilloma virus is HPV-16, HPV-18, HPV-31, HPV-33, HPV-34, HPV-35, HPV-39, HPV-45, HPV-51, HPV-52, HPV-56, HPV-58, HPV-59, HPV-65, HPV-66, HPV-68, HPV-69, or a combination of two or more thereof.

Embodiment 5. The method of Embodiment 3, wherein the human papilloma virus is HPV-16, HPV-18, or a combination thereof.

Embodiment 6. The method of any one of Embodiments 3 to 5, wherein the cancer is head and neck cancer, cervical cancer, penile cancer, vaginal cancer, anal cancer, vulvar cancer, bladder cancer, or breast cancer.

Embodiment 7. The method of Embodiment 6, wherein the head and neck cancer is oropharyngeal cancer.

Embodiment 8. The method of Embodiment 1 or 2, wherein the oncovirus is hepatitis B.

Embodiment 9. The method of Embodiment 8, wherein the cancer is liver cancer, stomach cancer, colorectal cancer, oral cavity cancer, pancreatic cancer, or lymphoma.

Embodiment 10. The method of Embodiment 9, wherein the lymphoma is non-Hodgkin lymphoma.

Embodiment 11. The method of Embodiment 1 or 2, wherein the oncovirus is hepatitis C.

Embodiment 12. The method of Embodiment 11, wherein the cancer is liver cancer, non-Hodgkin lymphoma, or head and neck cancer.

Embodiment 13. The method of Embodiment 12, wherein the head and neck cancer is an oral cavity cancer, oropharyngeal cancer, or laryngeal cancer.

Embodiment 14. The method of Embodiment 1 or 2, wherein the oncovirus is cytomegalovirus.

Embodiment 15. The method of Embodiment 14, wherein the cancer is colon cancer, glioma, prostate cancer, breast cancer, salivary gland cancer, or neuroblastoma.

Embodiment 16. The method of Embodiment 15, wherein the breast cancer is inflammatory breast cancer.

Embodiment 17. The method of Embodiment 1 or 2, wherein the oncovirus is Epstein-Barr virus.

Embodiment 18. The method of Embodiment 17, wherein the cancer is head and neck cancer, lymphoma, breast cancer, or stomach cancer.

Embodiment 19. The method of Embodiment 18, wherein the head and neck cancer is nasopharyngeal cancer.

Embodiment 20. The method of Embodiment 18, wherein the lymphoma is Burkitt lymphoma, Hodgkin lymphoma, non-Hodgkin lymphoma, or nasal type extranodal NK/T-cell lymphoma.

Embodiment 21. The method of Embodiment 1 or 2, wherein the oncovirus is human immunodeficiency virus.

Embodiment 22. The method of Embodiment 21, wherein the cancer is melanoma, Hodgkin lymphoma, anal cancer, lung cancer, liver cancer, testicular cancer, oral cavity cancer, or throat cancer.

Embodiment 23. The method of Embodiment 1 or 2, wherein the oncovirus is human herpes virus-8.

Embodiment 24. The method of Embodiment 23, wherein the cancer is Kaposi's sarcoma or lymphoma.

Embodiment 25. The method of Embodiment 24, wherein the lymphoma is primary effusion lymphoma or multicentric Castleman disease.

Embodiment 26. The method of Embodiment 1 or 2, wherein the virus is merkel cell polyomavirus.

Embodiment 27. The method of Embodiment 26, wherein the cancer is merkel cell carcinoma.

Embodiment 28. The method of Embodiment 1 or 2, wherein the oncovirus is John Cunningham polyomavirus.

Embodiment 29. The method of Embodiment 28, wherein the cancer is gastrointestinal cancer.

Embodiment 30. The method of Embodiment 1 or 2, wherein the virus is BK polyomavirus.

Embodiment 31. The method of Embodiment 30, wherein the cancer is lung cancer, pancreatic cancer, liver cancer, urogenital tract cancer, head and neck cancer, rhabdomyosarcoma, Kaposi's sarcoma, brain cancer, or bladder cancer.

Embodiment 32. The method of Embodiment 1 or 2, wherein the oncovirus is adenovirus.

Embodiment 33. The method of Embodiment 32, wherein the cancer is brain cancer, gastrointestinal cancer, lymphoma.

Embodiment 34. The method of Embodiment 33, wherein the brain cancer is glioblastoma, oligodendroglioma, or ependymomas.

Embodiment 35. The method of Embodiment 1 or 2, wherein the oncovirus is human T-cell leukemia virus type-1.

Embodiment 36. The method of Embodiment 35, wherein the cancer is adult T-cell leukemia/lymphoma.

Embodiment 37. The method of Embodiment 1 or 2, wherein the virus is human T-cell leukemia virus type-2.

Embodiment 38. The method of Embodiment 37, wherein the cancer is cutaneous T-cell lymphoma.

Embodiment 39. The method of any one of Embodiments 1 to 38, wherein the anti-CD73 compound is an anti-CD73 antibody.

Embodiment 40. The method of any one of Embodiments 1 to 39, wherein the anti-CD73 compound is immunostimulatory.

Embodiment 41. The method of any one of Embodiments 1 to 40, wherein the anti-CD73 compound is capable of (i) binding a CD73 protein, (ii) inhibiting CD73 catalytic activity, (iii) binding to and activating a B cell, (iv) binding to a B cell and inducing expression of an activation marker, (v) binding to a B cell and inducing maturation of the B cell into a plasma cell, (vi) binding to a B cell and inducing antibody secretion to the oncovirus, (vi) binding to a B cell and inducing memory B cells, or (vii) a combination of two or more of the foregoing.

Embodiment 42. The method of any one of Embodiments 39 to 41, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

Embodiment 43. The method of Embodiment 42, wherein the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and the CDR H3 has a sequence of SEQ ID NO:6.

Embodiment 44. The method of Embodiment 42 or 43, wherein the humanized heavy chain variable region comprises the sequence of SEQ ID NO:7.

Embodiment 45. The method of any one of Embodiments 42 to 44, wherein the humanized light chain variable region comprises the sequence of SEQ ID NO:8.

Embodiment 46. The method of any one of Embodiments 42 to 45, the anti-CD73 antibody comprises a glutamine at a position corresponding to Kabat position 297.

Embodiment 47. The method of any one of Embodiments 39 to 46, wherein the anti-CD73 antibody is an IgG.

Embodiment 48. The method of any one of Embodiments 39 to 46, wherein the anti-CD73 antibody is an IgG1 or an IgG4.

Embodiment 49. The method of any one of Embodiments 39 to 48, wherein the anti-CD73 antibody is a Fab′ fragment or a single chain antibody (scFv).

Embodiment 50. The method of any one of Embodiments 39 to 49, wherein the anti-CD73 antibody is capable of binding a CD73 antigen with an equilibrium dissociation constant (K_{D}) from about 0.3 to about 25 nM.

Embodiment 51. The method of any one of Embodiments 39 to 50, wherein the anti-CD73 antibody is capable of binding a CD73 antigen at a pH from about 6.0 to about 7.0.

Embodiment 52. The method of Embodiment 39, wherein the anti-CD73 antibody is oleclumab.

Embodiment 53. The method of Embodiment 39, wherein the anti-CD73 antibody is uliledlimab.

Embodiment 54. The method of Embodiment 39, wherein the anti-CD73 antibody is AD2.

### Example

Patients with oropharyngeal cancer (i.e., head and neck cancer) positive for the human papilloma virus (e.g., HPV-16 and/or HPV-18) will be treated with CPI-006. The results will show that the patients treated with CPI-006 had a longer time to tumor progression and greater evidence of tumor regression compared to patients who received a placebo.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Informal Sequence Listing

SEQ ID NO: 1:
RASKNVSTSGYSYMH
SEQ ID NO: 2:
LASNLES
SEQ ID NO: 3:
QHSRELPFT
SEQ ID NO: 4:
GYTFTSYWIT
SEQ ID NO: 5:
PGSGNTNYNEKFKT
SEQ ID NO: 6:
EGGLTTEDYALDY
SEQ ID NO: 7:
QVQLVQSGAEVEKPGASVKVSCKASGYTFTSYWITWVRQAPGQGLEWMG
DIYPGSGNTNYNEKFKTRVTITADKSTSTAYMELSSLRSEDTAVYYCAK
EGGLTTEDYALDYWGQGTLVTV
SEQ ID NO: 8:
EIVLTQSPATLSLSPGERATLSCRASKNVSTSGYSYMHWYQQKPGQAPR
LLIYLASNLESGIPPRFSGSGYGTDFTLTINNIESEDAAYYFCQHSREL
PFTFGQGTKVEIK
SEQ ID NO: 9
MEWSWVFLFFLSVTTGVHSQVQLVQSGAEVEKPGASVKVSCKASGYTFT
SYWITWVRQAPGQGLEWMGDIYPGSGNTNYNEKFKTRVTITADKSTSTA
YMELSSLRSEDTAVYYCAKEGGLTTEDYALDYWGQGTLVTVSSASTKGP
SVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHED
PEVKFNWYVDGVEVHNAKTKPREEQYQSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGK
SEQ ID NO: 10
MSVPTQVLGLLLLWLTDARCEIVLTQSPATLSLSPGERATLSCRASKNV
STSGYSYMHWYQQKPGQAPRLLIYLASNLESGIPPRFSGSGYGTDFTLT
INNIESEDAAYYFCQHSRELPFTFGQGTKVEIKRTVAAPSVFIFPPSDE
QLKSGTASVVCLLNNFYPREAKVQWKVDNALQSGNSQESVTEQDSKDST
YSLSSTLTLSKADYEKHKVYACEVTHQGLSSPVTKSFNRGEC

What is claimed is:

1. A method of treating an oncovirus positive cancer in a patient in need thereof, the method comprising administering to the patient an effective amount of an anti-CD73 compound.

2. A method of treating a cancer in a patient in need thereof, the method comprising:
(i) detecting an oncovirus in a cancer cell obtained from the patient; and
(ii) administering to the patient an effective amount of an anti-CD73 compound.

3. The method of claim 1, wherein the oncovirus is human papilloma virus.

4. The method of claim 3, wherein the human papilloma virus is HPV-16, HPV-18, HPV-31, HPV-33, HPV-34, HPV-35, HPV-39, HPV-45, HPV-51, HPV-52, HPV-56, HPV-58, HPV-59, HPV-65, HPV-66, HPV-68, HPV-69, or a combination of two or more thereof.

5. The method of claim 3, wherein the human papilloma virus is HPV-16, HPV-18, or a combination thereof.

6. The method of claim 4, wherein the cancer is head and neck cancer, cervical cancer, penile cancer, vaginal cancer, anal cancer, vulvar cancer, bladder cancer, or breast cancer.

7. The method of claim 6, wherein the head and neck cancer is oropharyngeal cancer.

8. The method of claim 1, wherein the oncovirus is hepatitis B.

9. The method of claim 8, wherein the cancer is liver cancer, stomach cancer, colorectal cancer, oral cavity cancer, pancreatic cancer, or lymphoma.

10. The method of claim 9, wherein the lymphoma is non-Hodgkin lymphoma.

11. The method of claim 1, wherein the oncovirus is hepatitis C.

12. The method of claim 11, wherein the cancer is liver cancer, non-Hodgkin lymphoma, or head and neck cancer.

13. The method of claim 12, wherein the head and neck cancer is an oral cavity cancer, oropharyngeal cancer, or laryngeal cancer.

14. The method of claim 1, wherein the oncovirus is cytomegalovirus.

15. The method of claim 14, wherein the cancer is colon cancer, glioma, prostate cancer, breast cancer, salivary gland cancer, or neuroblastoma.

16. The method of claim 15, wherein the breast cancer is inflammatory breast cancer.

17. The method of claim 1, wherein the oncovirus is Epstein-Barr virus.

18. The method of claim 17, wherein the cancer is head and neck cancer, lymphoma, breast cancer, or stomach cancer.

19. The method of claim 18, wherein the head and neck cancer is nasopharyngeal cancer.

20. The method of claim 18, wherein the lymphoma is Burkitt lymphoma, Hodgkin lymphoma, non-Hodgkin lymphoma, or nasal type extranodal NK/T-cell lymphoma.

21. The method of claim 1, wherein the oncovirus is human immunodeficiency virus.

22. The method of claim 21, wherein the cancer is melanoma, Hodgkin lymphoma, anal cancer, lung cancer, liver cancer, testicular cancer, oral cavity cancer, or throat cancer.

23. The method of claim 1, wherein the oncovirus is human herpes virus-8.

24. The method of claim 23, wherein the cancer is Kaposi's sarcoma or lymphoma.

25. The method of claim 24, wherein the lymphoma is primary effusion lymphoma or multicentric Castleman disease.

26. The method of claim 1, wherein the virus is merkel cell polyomavirus.

27. The method of claim 26, wherein the cancer is merkel cell carcinoma.

28. The method of claim 1, wherein the oncovirus is John Cunningham polyomavirus.

29. The method of claim 28, wherein the cancer is gastrointestinal cancer.

30. The method of claim 1, wherein the virus is BK polyomavirus.

31. The method of claim 30, wherein the cancer is lung cancer, pancreatic cancer, liver cancer, urogenital tract cancer, head and neck cancer, rhabdomyosarcoma, Kaposi's sarcoma, brain cancer, or bladder cancer.

32. The method of claim 1, wherein the oncovirus is adenovirus.

33. The method of claim 32, wherein the cancer is brain cancer, gastrointestinal cancer, lymphoma.

34. The method of claim 33, wherein the brain cancer is glioblastoma, oligodendroglioma, or ependymomas.

35. The method of claim 1, wherein the oncovirus is human T-cell leukemia virus type-1.

36. The method of claim 35, wherein the cancer is adult T-cell leukemia/lymphoma.

37. The method of claim 1, wherein the virus is human T-cell leukemia virus type-2.

38. The method of claim 37, wherein the cancer is cutaneous T-cell lymphoma.

39. The method of claim 1, wherein the anti-CD73 compound is an anti-CD73 antibody.

40. The method of claim 1, wherein the anti-CD73 compound is immunostimulatory.

41. The method of claim 1, wherein the anti-CD73 compound is capable of:
(i) binding a CD73 protein,
(ii) inhibiting CD73 catalytic activity,
(iii) binding to and activating a B cell,
(iv) binding to a B cell and inducing expression of an activation marker,
(v) binding to a B cell and inducing maturation of the B cell into a plasma cell,
(vi) binding to a B cell and inducing antibody secretion to the oncovirus,
(vi) binding to a B cell and inducing memory B cells, or
(vii) a combination of two or more of the foregoing.

42. The method of claim 39, wherein the anti-CD73 antibody comprises a 1E9 antibody CDR L1, a 1E9 antibody CDR L2, a 1E9 antibody CDR L3, a 1E9 antibody CDR H1, a 1E9 antibody CDR H2, and a 1E9 antibody CDR H3.

43. The method of claim 42, wherein the CDR L1 has a sequence of SEQ ID NO:1, the CDR L2 has a sequence of SEQ ID NO:2, the CDR L3 has a sequence of SEQ ID NO:3; the CDR H1 has a sequence of SEQ ID NO:4, the CDR H2 has a sequence of SEQ ID NO:5, and the CDR H3 has a sequence of SEQ ID NO:6.

44. The method of claim 42, wherein the humanized heavy chain variable region comprises the sequence of SEQ ID NO:7.

45. The method of claim 42, wherein the humanized light chain variable region comprises the sequence of SEQ ID NO:8.

46. The method of claim 42, the anti-CD73 antibody comprises a glutamine at a position corresponding to Kabat position 297.

47. The method of claim 39, wherein the anti-CD73 antibody is an IgG.

48. The method of claim 39, wherein the anti-CD73 antibody is an IgG1 or an IgG4.

49. The method of claim 39, wherein the anti-CD73 antibody is a Fab′ fragment or a single chain antibody (scFv).

50. The method of claim 39, wherein the anti-CD73 antibody is capable of binding a CD73 antigen with an equilibrium dissociation constant (K_{D}) from about 0.3 to about 25 nM.

51. The method of claim 39, wherein the anti-CD73 antibody is capable of binding a CD73 antigen at a pH from about 6.0 to about 7.0.

52. The method of claim 39, wherein the anti-CD73 antibody is oleclumab.

53. The method of claim 39, wherein the anti-CD73 antibody is uliledlimab.

54. The method of claim 39, wherein the anti-CD73 antibody is AD2.


SEQUENCE LISTING (10 sequences)

SEQ ID NO: 1 - protein, 15 aa, Artificial Sequence
  Synthetic Polypeptide
RASKNVSTSGYSYMH

SEQ ID NO: 2 - protein, 7 aa, Artificial Sequence
  Synthetic Polypeptide
LASNLES

SEQ ID NO: 3 - protein, 9 aa, Artificial Sequence
  Synthetic Polypeptide
QHSRELPFT

SEQ ID NO: 4 - protein, 10 aa, Artificial Sequence
  Synthetic Polypeptide
GYTFTSYWIT

SEQ ID NO: 5 - protein, 14 aa, Artificial Sequence
  Synthetic Polypeptide
PGSGNTNYNEKFKT

SEQ ID NO: 6 - protein, 13 aa, Artificial Sequence
  Synthetic Polypeptide
EGGLTTEDYALDY

SEQ ID NO: 7 - protein, 120 aa, Artificial Sequence
  Synthetic Polypeptide
QVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTV

SEQ ID NO: 8 - protein, 111 aa, Artificial Sequence
  Synthetic Polypeptide
EIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIK

SEQ ID NO: 9 - protein, 471 aa, Artificial Sequence
  Synthetic Polypeptide
MEWSWVFLFFLSVTTGVHSQVQLVQSGAEVEKPGASVKVSCKASGYTFTSYWITWVRQAPGQGLEWMGDIYPGSGNTNYNEKFKTRVTITADKSTSTAYMELSSLRSEDTAVYYCAKEGGLTTEDYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 238 aa, Artificial Sequence
  Synthetic Polypeptide
MSVPTQVLGLLLLWLTDARCEIVLTQSPATLSLSPGERATLSCRASKNVSTSGYSYMHWYQQKPGQAPRLLIYLASNLESGIPPRFSGSGYGTDFTLTINNIESEDAAYYFCQHSRELPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC